(19) United States
(12) Patent Application Publication
(10) Pub. No.: US 2025/0093343 A1
(43) Pub. Date: Mar. 20, 2025

(54) IMPROVED EXOSOME PROFILING FOR THERAPY AND DIAGNOSIS

(71) Applicant: NeuroDex, Inc., Natick, MA (US)

(72) Inventors: Erez Eitan, Arlington, MA (US); Olga Volpert, Waltham, MA (US)

(21) Appl. No.: 18/720,619

(22) PCT Filed: Dec. 19, 2022
(86) PCT No.: PCT/IB2022/062442

§ 371 Date: Jun. 14, 2024

Related U.S. Application Data
(60) Provisional application No. 63/291,405, filed on Dec. 19, 2021.
(60) Provisional application No. 63/291,407, filed on Dec. 19, 2021.

(51) Int. Cl. G01N 33/543; A61K 9/51; B82Y 5/00; G01N 21/64; G01N 33/58; G01N 33/68

(52) U.S. Cl. CPC G01N 33/54333; A61K 9/5176; A61K 9/5192; G01N 21/6428; G01N 33/582; G01N 33/6872; G01N 33/6896; B82Y 5/00; G01N 2333/70596

(57) ABSTRACT

The invention provides assays and methods for characterization of specific extracellular vesicle (EV) populations, and to the preparation of improved EV compositions for therapy and diagnosis. Specifically, the invention in embodiments thereof provides methods and kits for analyzing blood-derived samples, as well as to the production and use of tissue-derived circulating EV populations characterized by unexpectedly small dimensions and advantageous properties.

### FIELD OF THE INVENTION

The invention relates to assays and methods for simultaneous characterization of extracellular vesicle (EV) populations, and to the preparation of improved EV compositions for therapy and diagnosis.

### BACKGROUND OF THE INVENTION

Extracellular vesicles (EV) are released by all cells in the body, via diverse mechanisms including outward budding of the plasma membrane, exocytosis (microvesicles) and inward budding of endosomal membranes (exosomes) (Rak 2010. Semin Thromb Hemost 36:888-906; Colombo et al. 2014. Annu Rev Cell Dev Biol 30:255-289). Regardless of their biogenesis pathway, it is widely accepted that most biological fluids (blood, urine, cerebrospinal fluid, etc.) contain EV arising from multiple cell types. Further, a large body of experimental evidence shows that the molecular composition of EVs closely reflects that of the cells and tissues of their origin (He et al. 2018 Theranostics 8:237-255). Additionally, EV surface proteins may also reflect their cellular origin, and potentially elucidate the biogenesis pathway, destination, and function of the EV (Colombo et al. 2014. Annu Rev Cell Dev Biol 30: 255-289).

EV comprise a heterogenous group of particles, varying in subcellular origin, size, and composition. The functionality of EV has hitherto been perceived of as migrating between the releasing and accepting cells, the latter being where endocytosis and (intraluminal) cargo delivery trigger a phenotypic response. Although this communication paradigm is promising and supported by literature, EV can also act in an autocrine fashion or have other ‘delivery-independent’ extracellular functions such as modulation of the extracellular matrix (ECM), interactions with plasma membrane receptors transporting EV-resident proteins to recipient cells.

EV exhibit broad size distribution that can typically range from 30-5000 nm. EV size has been suggested to be a defrentiator of biogensis. For example exosomes have been defined as vesicles of 30 nm to 150 nm in diameter originating from endosomes/multivesicular bodies by reverse-inward budding, microvesicles have been defined as particles ranging between 150 nm to 1000 nm and produced through the outward budding of the plasma membrane, and apoptotic bodies (induced by vesiculation of an entire cell) have been identified as ranging between 500 nm and 5000 nm in diameter. However, other evidence suggest that these definitions are not precise, and that there is a significant overlap between the different EV types. Therefore, molecular techniques for EV profiling are needed to further define and characterize EV populations.

The development of FACS analysis to characterize surface proteins on immune cells revolutionized the field of immunology and the developing ability to extend this capacity towards analysis of EV may prove to be highly advantageous. In response to this opportunity gap, a number of assays was developed for characterization of intact EV (Kanwar et al. 2014 Lab Chip 14:1891-1900; Ohmichi et al. 2019 Parkinsonism Relat Disord 61:82-87; Ter-Ovanesyan et al. 2021 bioRxiv: 2020.2010.2013.337881). All these assays are based on a sandwich principle, wherein the sandwich is formed by an EV and two antibodies targeting distinct proteins on the EV surface. To generate a signal, both proteins should be inter-connected, presumably via the EV membrane. This principle can be incorporated into multiple immunoassay systems including traditional ELISA, Mesoscale Discovery electrochemiluminescence, SIMOA, FACS, SPR and many others. However, most of these methods are unsuitable for multiplexing and thus disallow simultaneous assessment of multiple EV surface proteins (Nolan and Duggan 2018 Methods Mol Biol 1678:79-92; Chiang and Chen 2019 J Biomed Sci 26:9; Kurian et al. 2021 Mol Biotechnol 63:249-266). The exception is the MACSPlex FACS Exosome Kit (Miltenyi Biotech, cat. No 130-108-813), which enables the detection of multiple EV surface epitopes (a total of 37). However, its capacity for simultaneous assessment is limited by the number of compatible fluorescent dyes and dye interference. In addition, this is an arduous methodology that requires EV enrichment prior to analysis and a skilled operator.

Microsphere-based suspension array technologies, such as the Luminex® xMAP® system, offer high-throughput detection of protein and nucleic acid targets in multiple assay chemistries. Attempt to utilize such technologies in a variety of applications that range from transplant medicine, biomarker discovery and validation, pathogen detection, drug discovery, vaccine development, personalized medicine, neurodegeneration, and cancer research, have been reported (Graham et al., 2019. Methods 158:2-11). For example, the use of nucleic acid probes that are affixed to fluorescent microbeads to be analyzed using a Luminex detector, is suggested in U.S. Pat. Nos. 8,232,058 and 7,642,348.

WO2021231720 discloses methods of using EV to detect complement activation, and uses thereof to assess and/or monitor treatment of a complement-mediated disease. In particular, provided is an immunoprecipitation bead-based immunocapture protocol with immunofluorescent detection, comprising a combination of EV enrichment (using pan-EV antibodies) and immunocapture of tissue-specific biomarkers present on the surface of shed EV, prior to detecting complement activation.

The high bioavailability and unique characteristics of EV have garnered them increasing focus as potential diagnostic and therapeutic applications. Several groups have described methods for the EV-based diagnosis of diseases including cancer, neurodegenerative disorders, viral infections and more. For example, Szajnik et al. (Gynecol. Obstet. 2013, Suppl 4:003) suggested a potential for differentiation between ovarian cancer patients and patients with benign tumors or healthy controls, based on characterization of the cargoes and epitopes of tumor released exosomes. EV-based biomarkers and their potential use are disclosed in U.S. Pat. No. 9,958,460 and WO2017193115. Various other publications involving the isolation and/or analysis of vesicles from biological samples include e.g. US20190219578, US20180340945, US20190361037, US20180080945, US20190137517 and WO2016172598.

EV have been increasingly used as therapeutic agents, encompassing a scope of pathological situations, including but not limited to treatment against infectious pathogens, mediation of the therapeutic effects of mesenchymal stem/stromal cells (MSC), modulation of the immune response and more. The characterization of EV-based therapeutics comprises a multi-step process, complicated by several technical issues, including the isolation and capture of the relevant EV in an efficient, convenient, and cost-conscious manner.

In particular, the distinctive attributes of EV and their participation in a host of physiological processes have made them attractive candidates as carriers for targeted drug delivery and gene therapy. Various studies suggest that EV have several advantages over conventional synthetic carriers, but despite extensive research, clinical translation of EV-based therapies remains challenging.

U.S. Pat. No. 11,111,475 is directed to a method of treating a human subject who has suffered a stroke, comprising administration of isolated EV derived from non-transformed human neural progenitor cells intravenously or intranasally. These progenitor cell-derived EV may be of different sizes, for example ranging from 20 nm to 150 nm, and may optionally further comprise an agent selected from the group consisting of small molecule, an antisense oligonucleotide, siRNA, an exogenous peptide, an exogenous protein, and an antibody. The publication discloses isolation of the EV from culture medium of cultured neural progenitor cells that were produced from pluripotent stem cells (e.g. human embryonic stem cells) or induced pluripotent stem cells (iPSCs)). Several other examples of therapeutic use of EV-based compositions include WO2020257710, U.S. Pat. Nos. 10,912,736, and 10,590,417.

Volpert et al., 2022, to the inventor and coworkers, published after the priority date of the present application, report a modification of Luminex assay for characterization of extracellular vesicle populations in biofluids (Volpert et al., bioRxiv preprint posted Jan. 12, 2022).

It is quite evident from the diversity of the studies regarding the diagnostic, prognostic and therapeutic efficacy of EV-based assays and treatments that much is still desired in terms of sensitivity, specificity and technical utility of said assays. In particular, the development of improved assays and methods for simultaneous characterization of EV populations, enabling reduction of the sample size required for analysis, and minimizing the time and/or technical complexity associated with currently available assays, would be highly advantageous. In addition, the production of EV compositions characterized by improved pharmacokinetic properties and/or tissue penetration would be beneficial in the development of improved drug-delivery platforms and other EV-based therapies.

### SUMMARY OF THE INVENTION

The invention relates to assays and methods for simultaneous characterization of extracellular vesicle (EV) populations, and to the preparation of improved EV compositions for therapy and diagnosis. Specifically, the invention in embodiments thereof provides methods and kits for analyzing blood-derived samples, as well as to the production and use of tissue-derived circulating EV populations exhibiting advantageous properties, characterized by unexpectedly small dimensions of less than 30 nm in diameter.

The invention is based, in part, on the development of an improved assay for multiplexed detection and analysis of EV-associated biomarkers, directly from low volumes of biofluids such as blood-derived samples. Unexpectedly, EV populations of various cellular origins were found, using the Luminex-compatible assay developed, to exhibit unique expression profiles of tetraspanin markers characteristic of each population. In addition, the developed Luminex-compatible assay enabled multiplexed detection, quantification and analysis of a plurality of synaptic proteins presented on the surface of EVs, using very small volumes of blood-derived samples. Advantageously, the developed assay is much more simple and accurate compared to hitherto described methods for measuring synaptic proteins in blood-derived samples, which mainly rely on immunoprecipitation followed by mass spectrometry. The assay disclosed herein is therefore particularly suitable and useful for clinical diagnostic applications.

The invention is further based, in part, on the surprising discovery, that certain populations of circulating EV, such as those of neuron, and macrophage origin, are markedly smaller in size than the average size of circulating exosomes or hitherto-identified EV populations (such as erythrocyte-derived EV). In particular, disclosed herein for the first time are isolated EV of a neuronal origin that are <30 nm in diameter; these EV were characterized by a particle size distribution ranging from about 15-25 nm as evaluated by size exclusion chromatography (SEC) followed by transmission electron microscopy (TEM). In contradistinction, EV populations secreted by cultured neural cells (differentiated ex vivo from induced pluripotent stem cells), did not share the unique size properties of their circulating counterparts, despite the observation of similar expression profiles of neural markers and tetraspanins in the two populations. Rather, EV from cultured neural cells exhibited properties consistent with average diameter of 100-200 nm, similar to the evaluated dimensions of erythrocyte-derived EV. In addition, the EV isolated from late SEC fractions (corresponding to reduced EV diameters) were also found to be characterized by a unique lipid profile, to contain tissue-specific RNA markers and to exhibit advantageous properties including enhanced stability as evaluated by detergent resistance.

Accordingly, disclosed herein are methods and assays for profiling and characterizing tissue-specific EV populations from blood-derived samples, based on newly identified markers and size distribution, and utilizing improved methodology allowing multiplexed measurements in low (e.g. <75 μl) sample volumes. In addition, disclosed herein are newly identified populations of tissue-specific, blood-circulating EV (herein designated “nano-EV”), that are distinguishable by their physico-chemical properties and tissue specificity from other blood-circulating EV populations. Further disclosed herein are methods for the preparation of non-naturally occurring EV compositions comprising purified (e.g. pharmaceutical-grade purity of) nano-EV, and their improved diagnostic and therapeutic uses, including in drug delivery and gene therapy.

According to one aspect, the present invention provides an extracellular vesicle (EV) preparation comprising a substantially purified population of EVs isolated from serum or plasma derived from cells other than blood cells, the population is characterized by a size distribution of 10-25 nanometers (nm) in diameter and by a marker profile corresponding to differentiated solid tissue cells.

As used herein, a “substantially purified” population of EVs isolated from serum or plasma indicates that the EVs are at least 80% separated from other plasma/serum components, for example at least 85%, at least 90%, at least 95% separated from other plasma/serum components. The EVs isolated from serum or plasma, also referred to herein as circulating EVs, are derived from cells other than blood cells, namely, other than erythrocytes, platelets and leukocytes.

In some embodiments, the EVs are 15-25 nm in diameter. In additional embodiments, the EVs are 15-20 nm in diameter. In yet additional embodiments, the EVs are 10-15 nm in diameter.

In some embodiments, the EVs comprise at least one type of EV selected from neural-derived EV and tissue macrophage-derived EV. In some embodiments, the EVs comprise neural-derived EVs and tissue macrophage-derived EVs. In some embodiments, the population of EVs is characterized by surface display of GAP43, CD171 and/or CD68; and lack of CD235a. In some embodiments, the population of EVs is characterized by a membrane lipid composition according to Table A.

In some embodiments, the EVs are obtained from the plasma or serum sample by a process comprising a step of size selection for EVs that are under 30 nanometers (nm) in diameter, and isolation of the selected EVs. In some embodiments, the step of size selection comprises at least one of size exclusion chromatography, nanoporous membrane filtration, deterministic lateral displacement sorting, dielectrophoretic isolation, acoustic fractionation, and differential centrifugation. In some embodiments, the process further comprises immunoaffinity purification and/or analysis of the EV population.

In some embodiments, the EVs are loaded with an exogenous cargo. In some embodiments, the exogenous cargo is a therapeutic agent.

In some embodiments, the EVs are modified to display a targeting agent.

In some embodiments, the purity of the EV population is a pharmaceutical-grade purity. In some embodiments, the preparation is formulated in the form of a pharmaceutical composition, further comprising a pharmaceutically acceptable carrier, excipient or diluent. In some embodiments, the preparation is for use in therapy. In some embodiments, the therapy comprises delivery of an exogenously loaded therapeutic agent to a target cell or tissue of a subject in need thereof. In additional embodiments, the preparation is for use in diagnosis.

According to a further aspect, the present invention provides a method of analyzing a plasma or serum sample, comprising:
a. providing a capture system, comprising at least three populations of distinct fluorescence-labeled magnetic microspheres, wherein each microsphere population displays antibodies directed to a distinct target on the surface of an EV population of a distinct cellular origin;
b. providing 1-75 μl of an unprocessed plasma or serum sample, or a corresponding amount of intact EV;
c. incubating the sample with the capture system, under conditions such as to allow specific antigen-antibody binding while substantially maintaining the integrity of the EV membranes, to thereby provide distinct populations of EV-microsphere complexes corresponding to each target;
d. washing the EV-microsphere complexes using a magnetic device, under conditions enabling selective capturing of said complexes;
e. incubating the EV-microsphere complexes with a plurality of detection antibodies, each antibody directed to a distinct marker on the surface of the EV, under conditions such as to allow specific antigen-antibody binding while substantially maintaining the integrity of the EV membranes, wherein the plurality of detection antibodies is labeled by the same fluorescent marker;
f. washing the resulting labeled complexes using a magnetic device to remove excess reagents; and
g. subjecting the resulting complexes to a microfluidic device amenable for simultaneously detecting and quantifying fluorescent emission on a plurality of wave lengths, to thereby quantify the fluorescence emission levels and provide a separate assessment of the level of the surface targets corresponding to each of the EV populations,
wherein the method is performed using reagents and under conditions so as to retain said EVs in a substantially intact form.

In some embodiments, the method further comprises comparing the quantified levels to control levels.

In some embodiments, the plurality of detection antibodies is directed to a plurality of diagnostic markers. In some embodiments, the plurality of detection antibodies is directed to a plurality of tetraspanin markers. In some embodiments, the tetraspanin markers are selected from the group consisting of CD9, CD63 and CD81. In some particular embodiments, the plurality of detection antibodies comprises antibodies directed to CD9, antibodies directed to CD63 and antibodies directed to CD81.

In some embodiments, the targets of the capture system comprise at least one neural cell target and targets corresponding to at least two additional cellular origins selected from the group consisting of: bone, lung, tissue macrophage, lung macrophage, muscle, adipocyte, epithelium, endothelium, monocyte, microglia, megakaryocyte, T cell, erythrocyte, liver and oligodendrocyte. In some embodiments, the at least one neural cell target is selected from GAP43 and CD171, and the at least two additional targets comprise P2RY12 and CD68. In some embodiments, the at least two additional targets further comprise CD235a.

In some embodiments, the capture system comprises at least four populations of distinct fluorescence-labeled magnetic microspheres. In some embodiments, the capture system comprises at least five populations of distinct fluorescence-labeled magnetic microspheres. In some embodiments, the capture system comprises at least six populations of distinct fluorescence-labeled magnetic microspheres. In some embodiments, the capture system comprises at least seven populations of distinct fluorescence-labeled magnetic microspheres. In some embodiments, the capture system comprises at least eight populations of distinct fluorescence-labeled magnetic microspheres. Each microsphere population displays antibodies directed to a distinct target on the surface of an EV population of a distinct cellular origin.

In some embodiments, 1-50 μl of unprocessed plasma or serum are provided for the method.

According to a further aspect, there is provided herein a method for simultaneously measuring a plurality of synaptic proteins in a plasma or serum sample, the method comprising:
a. providing an unprocessed plasma or serum sample comprising extracellular vesicles (EV);
b. providing a capture system comprising at least two populations of fluorescence-labeled magnetic microspheres, each microsphere population displaying antibodies directed to a distinct EV surface target and labeled with a distinct fluorescent label, wherein the targets are synaptic proteins;
c. incubating the plasma or serum sample with the capture system under conditions that allow specific binding between the antibodies and the target synaptic proteins while substantially maintaining the integrity of the EV membranes, to thereby provide distinct populations of EV-microsphere complexes corresponding to each synaptic protein;
d. washing the EV-microsphere complexes using a magnetic device, under conditions enabling selective capturing of the complexes;
e. providing a plurality of detection antibodies directed to a plurality of canonical EV surface markers, wherein the plurality of detection antibodies is labeled, directly or indirectly, with the same fluorescent label;
f. incubating the captured EV-microsphere complexes with the plurality of detection antibodies under conditions that allow specific binding between the antibodies and EV surface markers while substantially maintaining the integrity of the EV membranes;
g. washing the resulting labeled complexes using a magnetic device to remove excess reagents; and
h. subjecting the labeled complexes to a microfluidic device amenable for simultaneously detecting and quantifying fluorescent emission on a plurality of wave lengths, to quantify fluorescence emission levels and provide a separate assessment of the level of the synaptic protein corresponding to each of the EV populations.

The method is performed using reagents and under conditions so as to retain the EVs in a substantially intact form

In some embodiments, the synaptic proteins are selected from the group consisting of glutamate receptor subunit 2 (GluR2), neurogranin (NRGN), growth-associated protein 43 (GAP43), postsynaptic density 95 (PSD95) and Syntaxin-1 (STXN-1).

In some embodiments, the at least two populations of fluorescence-labeled magnetic microspheres comprise a first population of fluorescence-labeled magnetic microspheres displaying antibodies directed to NRGN, and a second population of fluorescence-labeled magnetic microspheres displaying antibodies directed to GAP43.

In some embodiments, the at least two populations of fluorescence-labeled magnetic microspheres comprise a first population of fluorescence-labeled magnetic microspheres displaying antibodies directed to GluR2, a second population of fluorescence-labeled magnetic microspheres displaying antibodies directed to PSD95, and a third population of fluorescence-labeled magnetic microspheres displaying antibodies directed to STXN.

In some embodiments, the at least two populations of fluorescence-labeled magnetic microspheres comprise a first population of fluorescence-labeled magnetic microspheres displaying antibodies directed to NRGN, a second population of fluorescence-labeled magnetic microspheres displaying antibodies directed to GAP43, a third population of fluorescence-labeled magnetic microspheres displaying antibodies directed to GluR2, a fourth population of fluorescence-labeled magnetic microspheres displaying antibodies directed to PSD95, and a fifth population of fluorescence-labeled magnetic microspheres displaying antibodies directed to STXN.

In some embodiments, the canonical EV surface markers are tetraspanins. In some embodiments, the plurality of detection antibodies comprises at least three antibodies, each directed to a different tetraspanin. In some embodiments, the plurality of detection antibodies comprises antibodies directed to CD9, antibodies directed to CD63 and antibodies directed to CD81.

In some embodiments, the capture system further comprises a population of fluorescence-labeled magnetic microspheres displaying a non-specific control antibody, and providing assessment of the level of the synaptic proteins comprises normalizing the specific signal determined for each synaptic protein based on the non-specific control signal.

In some embodiments, the capture system further comprises a population of fluorescence-labeled magnetic microspheres displaying an antibody directed to a canonical EV surface marker, and providing assessment of the level of the synaptic proteins comprises normalizing the signal determined for each synaptic protein based on the signal of the canonical EV surface marker. In some embodiments, the canonical EV surface marker is selected from CD63, CD9, CD81, or a combination thereof.

In some embodiments, the method is carried out on 1-75 μl of plasma or serum (or a corresponding amount of intact EV).

In some embodiments, the plasma or serum sample is from a subject suspected of having a neurological disorder. In additional embodiments, the plasma or serum sample is from a subject suspected of having a neurodegenerative disorder. A subject according to the present invention is typically a human subject.

According to a further aspect, the present invention provides a kit for analyzing EVs from a blood-derived sample, comprising:
i) a capture system, comprising a first population of magnetic microspheres displaying an antibody directed to GAP43 or CD171, and labeled by a first combination of fluorophores, a second population of magnetic microspheres displaying an antibody directed to CD68, labeled by a second combination of fluorophores, and a third population of magnetic microspheres displaying an antibody directed to P2RY12, labeled by a third combination of fluorophores;
ii) a plurality of detection antibodies, each antibody directed to a distinct tetraspanin marker, wherein the plurality of detection antibodies is labeled, directly or indirectly, by the same fluorescent marker; and optionally
iii) reagents for performing the analysis under conditions so as to retain EVs in a substantially intact form.

In some embodiments, the capture system comprises a population of magnetic microspheres displaying an antibody directed to GAP43 and a population of magnetic microspheres displaying an antibody directed to CD171.

According to a further aspect, the present invention provides a kit for analyzing EVs from a blood-derived sample, comprising:
i) a capture system, comprising at least two populations of fluorescence-labeled magnetic microspheres, each microsphere population displaying antibodies directed to a distinct EV surface target and labeled with a distinct fluorescent label, wherein the targets are synaptic proteins;
ii) a plurality of detection antibodies directed to a plurality of canonical EV surface markers, wherein the plurality of detection antibodies is labeled, directly or indirectly, with the same fluorescent label; and optionally
iii) reagents for performing the analysis under conditions so as to retain EVs in a substantially intact form.

In some embodiments, the synaptic proteins are selected from the group consisting of glutamate receptor subunit 2 (GluR2), neurogranin (NRGN), growth-associated protein 43 (GAP43), postsynaptic density 95 (PSD95) and Syntaxin-1 (STXN-1).

In some embodiments, the kit comprises:
i) a capture system, comprising a first population of magnetic microspheres displaying an antibody directed to NRGN and labeled by a first combination of fluorophores, and a second population of magnetic microspheres displaying an antibody directed to GAP43 and labeled by a second combination of fluorophores; and
ii) a plurality of detection antibodies, each antibody directed to a distinct tetraspanin marker, wherein the plurality of detection antibodies is labeled, directly or indirectly, by the same fluorescent marker.

In some embodiments, the kit comprises:
i) a capture system, comprising a first population of magnetic microspheres displaying an antibody directed to GluR2 and labeled by a first combination of fluorophores, a second population of magnetic microspheres displaying an antibody directed to PSD95 and labeled by a second combination of fluorophores, and a third population of magnetic microspheres displaying an antibody directed to STXN and labeled by a third combination of fluorophores; and
ii) a plurality of detection antibodies, each antibody directed to a distinct tetraspanin marker, wherein the plurality of detection antibodies is labeled, directly or indirectly, by the same fluorescent marker.

In some embodiments, the kit comprises:
a first population of magnetic microspheres displaying antibodies directed to NRGN and labeled by a first combination of fluorophores, a second population of magnetic microspheres displaying antibodies directed to GAP43 and labeled by a second combination of fluorophores, a third population of magnetic microspheres displaying antibodies directed to GluR2 and labeled by a third combination of fluorophores, a fourth population of magnetic microspheres displaying antibodies directed to PSD95 and labeled by a fourth combination of fluorophores, and a fifth population of magnetic microspheres displaying antibodies directed to STXN and labeled by a fifth combination of fluorophores; and
ii) a plurality of detection antibodies, each antibody directed to a distinct tetraspanin marker, wherein the plurality of detection antibodies is labeled, directly or indirectly, by the same fluorescent marker.

In some embodiments, the detection antibodies comprise antibodies directed to CD9, antibodies directed to CD63 and antibodies directed to CD81.

In some embodiments, methods and kits as disclosed herein further comprise a population of fluorescence-labeled magnetic microspheres displaying a non-specific control antibody, for normalizing the specific signal determined for each target protein (e.g. synaptic protein) based on the non-specific control signal.

In some embodiments, methods and kits as disclosed herein further comprise a population of fluorescence-labeled magnetic microspheres displaying an antibody directed to a canonical EV surface marker, for normalizing the signal determined for each target protein (e.g. synaptic protein) based on the signal of the canonical EV surface marker. In some embodiments, the canonical EV surface marker is selected from CD63, CD9, CD81, or a combination thereof.

In some embodiments, the reagents are selected from the group consisting of:
(i) at least one binding buffer for incubating a sample with the capture system to thereby provide distinct populations of EV-microsphere complexes, the at least one binding buffer characterized by lack of detergents and by the presence of protease and/or phosphatase inhibitors;
(ii) at least one washing buffer, characterized by significantly enhanced salt concentrations compared to the at least one binding buffer; and
(iii) at least one binding buffer and at least one washing buffer as defined in (i) and (ii) above.

Other objects, features and advantages of the present invention will become clear from the following description and drawings.

### BRIEF DESCRIPTION OF THE DRAWINGS

FIGS. 1A-1J depict the performance of the developed assays as a function of various experimental parameters. FIG. 1A-net mean fluorescent intensity (MFI) obtained for loading the CD9 antibody on the capture beads by using staining with increasing amounts of PE-conjugated anti-mouse antibodies (denoted “Detection antibody” in the figure), presented for the various loading concentrations (μg\10\hat{ }6 beads), ranging from 0.5 to 5 μg/ml. FIG. 1B-1C—analysis of two random unprocessed plasma samples from healthy donors, using beads conjugated with capture antibodies against specific targets (CD9, GAP43, P2RY12, CD68 or the non-specific IgG used as negative control) and a cocktail of biotinylated detection antibodies for tetraspanin detection (CD9, CD63, and CD81 thereafter referred to as “panTSPN”). FIG. 1B-plasma sample obtained from a first healthy donor (denoted “plasma 1”); FIG. 1C plasma sample obtained from a second healthy donor (denoted “plasma 2”). FIG. 1D-net MFI measured with the panTSPN detection antibody cocktail on EV captured from plasma samples with either CD9, GAP43 or CD171-specific antibodies, in either intact form (“intact plasma”) or following EV depletion using PEG based precipitation (“EV depletion”) or detergent lysis (“Triton-X100, 2% final concentration, “Detergent”). White dots denote intact plasma, gray dotes denote samples treated with precipitation and hatched denote samples treated with detergent. P values by 2-way ANOVA were below 0.001 or below 0.0001, as indicated in the figure. FIG. 1E-1F—net MFI obtained against EV captured by CD171 or GAP43 antibodies in media conditioned by HEK293 cells overexpressing one of these neuronal antigen (CD171 or GAP43). FIG. 1E illustrates the detection of GAP43-positive EV by IEL beads appended with anti-GAP43 but not anti-CD171 antibodies. FIG. 1F illustrates the detection of CD171-positive EV by IEL beads appended with anti-CD171 but not anti-GAP43 antibodies; FIG. 1G-1J-plasma samples were analyzed using IEL beads specific for GAP43 (1G), CD171 (1H), CD235 (11) and CD68 (1J), paired with the indicated detection antibodies (panTSPN cocktail, GAP43, CD171, CD68 and CD235).

FIG. 2A-2G demonstrate simultaneous measurement of at least six surface EV proteins. FIG. 2A-net MFI measurements in serial plasma dilutions using two canonical EV surface markers (CD9 and CD81), or tissue-specific markers (macrophage marker CD68, erythrocyte marker CD235 (Glycophorin A), neuronal markers GAP43 and CD171). FIG. 2B-2F show detection of multiple tissue-specific and canonical EV surface markers, measured concurrently in both multiplexed and single-bead IEL assays. MFI obtained with anti-CD9, anti-CD81, anti-CD235, anti-GAP43 and anti-CD68 capture beads (FIGS. 2B-2F, respectively). FIG. 2G.—correlation between single-bead and multiplexed assays measuring net MFI obtained against CD9 in 25 random plasma samples.

FIG. 3A-3F depict cell type specific differences between the tetraspanin profiles of distinct EVs populations in blood plasma. FIG. 3A-whole plasma (10-50 ml/sample) was subjected to IEL with antibodies against an axonal marker, GAP43, followed by detection with biotinylated GAP43 antibodies or using panTSPN antibody cocktail. FIG. 3B—plasma IEL with GAP43 antibodies was followed by detection with individual TSPNs antibodies, namely: CD9, CD63 and CD81 antibodies. FIG. 3C-IEL for erythrocyte EV using a known marker CD235 glycophorin was followed by detection with individual TSPNs antibodies, namely: CD9, CD63 and CD81 antibodies. FIG. 3D—IEL for one of the EV markers, CD9, was followed by detection with CD235 and GAP43 antibodies. FIG. 3E, 3F-IEL for CD63 (E), CD81 (F) followed by GAP43 and CD235 detection shows strong positivity for GAP43 and the lack of detectable association with CD235.

FIG. 4A-4E illustrate IEL capture and detection analyses conducted on fractions of plasma EV following size exclusion chromatography (SEC). EVs from 10 ml plasma were precipitated using PEG-based approach, resuspended in 2 ml PBS, loaded onto QEV2 53 nm column (IZON Sciences) and 2 ml fractions were collected following the manufacturer's instructions. Protein concentration (A280, gray bars) was measured in each fraction, including 3 void fractions (denoted −2, −1 and 0) and 50 μl aliquots of each fraction analyzed by IEL as shown (black bars). The fractions with the peak concentrations of specific EVs (detected with panTSPN antibody cocktail) are indicated by arrows. (A) IEL for CD9, detection with panTSPN antibody cocktail. Protein elution is shown in grey. (B) IEL for erythrocyte marker CD235, detection with panTSPN antibody cocktail. (C) IEL for macrophage marker, CD68, detection with panTSPN antibody cocktail. (D) IEL for axonal marker, GAP43, detection with panTSPN antibody cocktail. (E) IEL for neuronal marker, CD171, detection with panTSPN antibody cocktail.

FIG. 5A-5G. Intact EV Luminex reveals a continuum of sizes amongst tetraspanin-positive structures (EVs) in blood plasma. (FIG. 5A-5D) Fractions collected from QEV2 column were subjected to IEL for axonal marker GAP43 and probed with the pan-TSPN antibody cocktail (A) or with the antibodies against CD9 (B), CD63 (C) and CD81 (D), respectively. (FIG. 5E-G) QEV2 fractions were subjected to IEL for CD9 (E), CD63 (F) and CD81 (G), and probed with antibodies against axonal marker, GAP43, or with panTSPN antibody cocktail, as shown.

FIG. 6A-6D. Plasma membrane disruption by Triton X-100 pre-treatment of plasma samples diminishes IEL signal. Plasma samples were concentrated by PEG-based precipitation, reconstituted in 1×PBS. The samples were then pre-treated with TX-100 (red bars) or sham buffer (blue bars) and subjected to size-exclusion chromatography (IZON qEV 2 column). The fractions were then subjected to IEL with (A) CD9, (B) CD235, (C) CD63 and (D) GAP43 beads paired with panTSPN detection cocktail.

FIG. 7A-7F. IEL analysis of neuronal cell conditioned medium. Serum-free medium conditioned by iPSC-derived neurons was collected, cleared by filtration via Amicon spin filter (100 kDa MWCO) reconstituted in 0.5 ml PBS, and processed by SEC using QEV original column (35 nm, ZON Sciences). Fractions (0.5 ml) were collected according to the manufacturer's instructions and protein concentration (A280) determined in each fraction (gray bars). All fractions (1-20, 50 ml per sample) were subjected to IEL with beads for (A) GAP43, (B) CD171 and (C) CD235 followed by detection with panTSPN antibody cocktail. Note the lack of CD235-positive EVs in the medium conditioned by iPSC neurons (specificity control). (D-F) IEL of the QEV fractions with beads for EV markers, CD9 (D), CD63 (E) and CD81 (F).

FIG. 8. Lipid composition of late and early SEC fractions. Plasma EV were concentrated as described in Methods and processed by SEC (qEV2, Izon). Fractions 2-5 and 8-13 were pooled, concentrated using Ammicon spin filter (100 kDA MWCO) and subjected to lipidomic analysis. Each fraction was analyzed in biological triplicate. The pie charts show proportional contribution (% of total lipid) for the indicated specific lipid classes in the early (left) and late fractions (right) is shown. “Exploding” sectors indicate lipids with higher proportional content. For minor lipid constituents (average content in early fractions <0.1%) see Table 8.

FIG. 9A-9B present transmission electron microscopy (TEM) images of EV stained with aqueous uranyl acetate in fractions 2-5 (FIG. 9A) and 8-14 (FIG. 9B).

FIG. 10A-10F present multiplexed measurement of five synaptic markers, glutamate receptor subunit 2 (GluR2, FIG. 10A), neurogranin (NRGN, FIG. 10B), growth-associated protein 43 (GAP43, FIG. 10C), postsynaptic density (PSD95, FIG. 10D) and Syntaxin-1 (STXN-1, FIG. 10E), and of the EV marker CD63 (FIG. 10F). The assay was performed in three formats: single plex (1-plex)-using capture beads for individual analytes; 6-plex-using a combination of capture beads for all six analytes; and 3-plex, wherein two parallel assays with two capture bead combinations (NRGN+GAP43+CD63 and GluR2+PSD95+STXN-1) were carried out.

FIG. 11A-11D show quantitative measurements using purified EV derived from cultured cells. A dilution curve was generated using concentrated purified EV released from cultured glioblastoma cells (U87 cell line, ATCC). The EV markers CD9 (FIG. 11A), CD81 (FIG. 11B) and CD63 (FIG. 11C) were measured in single plex (1-plex) and multiplexed (M-plex) formats with appropriate capture beads and panTSPN antibody cocktail for detection. Additionally, non-specific control beads decorated with isotype control antibodies (IgG) were added to the assay (FIG. 11D).

FIG. 12A-12E. Internal specificity control. Capture beads decorated with a non-specific isotype control antibody (IgG) were incorporated into the IEL assay. Single plex (1-plex) and multiplexed measurements were compared to ensure the lack of antibody interference. For each analyte, including CD63 (FIG. 12A), GAP43 (FIG. 12B), GluR2 (FIG. 12C), PSD95 (FIG. 12D) and NRGN (FIG. 12E), the specific signal exceeded the non-specific signal (IgG) by at least 10-fold.

### DETAILED DESCRIPTION OF THE INVENTION

The invention relates to assays and methods for simultaneous characterization of extracellular vesicle (EV) populations, and to the preparation of improved EV compositions for therapy and diagnosis. Specifically, the invention in embodiments thereof provides methods and kits for analyzing blood-derived samples, as well as to the production and use of tissue-derived circulating EV populations exhibiting advantageous properties, characterized by unexpectedly small dimensions of less than 30 nm in diameter.

The invention relates in embodiments thereof to methods for analyzing blood-derived samples (e.g. plasma and serum samples), herein referred to as “the analytical methods of the invention”. Unlike other biofluid sources such as urine, analysis of blood-derived samples is technically complicated by the existence of high concentrations of potential contaminants, including, but not limited to circulating proteins (e.g. albumin, fibrinogen and globulins), lipids (e.g. high-density lipoprotein, low-density lipoprotein, and triglycerides) and debris. In addition, blood-derived samples are characterized by high levels of vesicles from various origins, compounding the ability to detect tissue-specific EV markers without cross-contamination by other vesicles, especially in multiplexed assays. Indeed, analysis of blood-derived samples for EV markers typically involves preliminary EV enrichment steps (e.g. centrifugation, precipitation, size exclusion, filtration, and/or immunoprecipitation) in order to obtain purified EV in sufficient quantities compatible with existing measurement technologies, hence necessitating the use of higher sample volumes and additional labor.

In contradistinction, the present invention provides methods for analyzing EV from blood-derived samples which do not require isolating or enriching the EV prior to analysis, and which can be performed on very small sample volumes, between 1-100 μl, and even between 1-50 μl. The analytical methods in accordance with embodiments of the invention provide for multiplexed assays using Luminex or similar technologies. Typically and advantageously, the assay is employed (or marker assessment or quantification is performed) under conditions such that the EV remain substantially intact. For example, without limitation, analytical assays and methods of the invention advantageously employ the use of detergent-free buffers (e.g., when incubating a sample with a capture system in accordance with the invention, as described in further detail below) which may comprise protease and/or phosphatase inhibitors. In additional advantageous embodiments, assays and methods of the invention may involve enhancement of the salt concentration during the washing steps, while maintaining an essentially detergent-free environment.

In some embodiments the methods and assays employ high salt conditions (e.g. 50 mM-300 mM NaCl) to reduce non-specific interactions and improve the specificity of the measurement in complex blood-based biofluids. In some embodiments the methods and assays comprise a blocking strategy comprising competition with negatively charged peptides that reduces the interactions of the overall negatively charged EV, and thus improves the specificity of the measurement in a complex blood-based biofluid.

Blood-derived samples to be used in connection with the analytical methods of the invention advantageously comprise intact EV. In some embodiments, the sample is a plasma sample. In another embodiment, the sample is a serum sample. “Unprocessed/non-processed plasma or serum sample” as used herein indicates that the plasma or serum sample was not subjected to a process to isolate, separate or enrich for EV prior to contacting with the capture system as disclosed herein. The sample may be diluted or mixed with various assay reagents. Advantageously, as demonstrated herein, the analytical methods are amenable for use with low input volumes of biofluid samples. In some embodiments, 1-100 μl of plasma or serum are sufficient for the method, e.g., 1-75 μl, 1-50 μl of plasma or serum, less than 75 μl per data point (measurement) and typically 1-50 μl of plasma or serum samples are sufficient for the method. In another embodiment, the sample comprises 1-25, 15-25, or 10-20 μl (which may be diluted to a final volume compatible with the chosen assay, for example about 50 μl for Luminex-based assays), wherein each possibility represents a separate embodiment of the invention. However, it is to be understood that larger sample volumes can also be used in some embodiments in connection with the principles of the invention.

In an exemplary embodiment, analytical methods in accordance with the invention may comprise:
a. providing a capture system, comprising a plurality (e.g. at least three, four, five, six, seven or eight) populations of distinct fluorescence-labeled magnetic microspheres, wherein each microsphere population displays antibodies directed to distinct targets on the surface of EV populations of distinct cellular origins;
b. providing a blood-derived sample of the subject, the sample comprising less than 75 μl of non-processed plasma, or a corresponding amount of intact EV;
c. incubating the sample with the system, under conditions such as to allow specific antigen-antibody binding while substantially maintaining the integrity of the EV membranes, to thereby provide distinct populations of EV complexes with the microspheres (herein designated “EV-microsphere complexes”) corresponding to each target;
d. washing the EV-microsphere complexes using a magnetic device, under conditions enabling selective capturing of said complexes (e.g. to remove non-specific EV and soluble proteins);
e. incubating said EV-microsphere complexes with a plurality of detection antibodies, each antibody directed to a distinct marker on the surface of the EV, under conditions such as to allow specific antigen-antibody binding while substantially maintaining the integrity of the EV membranes, wherein the plurality of detection antibodies is labeled by the same fluorescent marker;
f. optionally and advantageously washing the resulting labeled complexes using a magnetic device to remove excess reagents (e.g., unbound antibody);
g. subjecting the resulting complexes to a microfluidic device amenable for detecting and simultaneously quantifying fluorescent emission on a plurality of wave lengths, to thereby quantify the fluorescence emission levels and provide a separate assessment of the level of the surface markers corresponding to each of the EV populations; and;
h. comparing the quantified levels to control levels (while typically considering the background levels of each measurement).

In some embodiments, there is provided a method for analyzing a blood-derived sample, the method comprising:
providing an unprocessed plasma or serum sample comprising extracellular vesicles (EV);
b. providing a capture system comprising at least two populations of fluorescence-labeled magnetic microspheres, each microsphere population displaying antibodies directed to a distinct EV surface target and labeled with a distinct fluorescent label, wherein the targets are synaptic proteins;
c. incubating the plasma or serum sample with the capture system under conditions that allow specific binding between the antibodies and the target synaptic proteins while substantially maintaining the integrity of the EV membranes, to thereby provide distinct populations of EV-microsphere complexes corresponding to each synaptic protein;
d. washing the EV-microsphere complexes using a magnetic device, under conditions enabling selective capturing of the complexes;
e. providing a plurality of detection antibodies directed to a plurality of canonical EV surface markers, wherein the plurality of detection antibodies is labeled, directly or indirectly, with the same fluorescent label;
f. incubating the captured EV-microsphere complexes with the plurality of detection antibodies under conditions that allow specific binding between the antibodies and EV surface markers while substantially maintaining the integrity of the EV membranes;
g. washing the resulting labeled complexes using a magnetic device to remove excess reagents; and
h. subjecting the labeled complexes to a microfluidic device amenable for simultaneously detecting and quantifying fluorescent emission on a plurality of wave lengths, to quantify fluorescence emission levels and provide a separate assessment of the level of the synaptic protein corresponding to each of the EV populations.

As used herein, the term “synaptic proteins” refers to membrane proteins that are known to be significantly enriched in the synapse space, compared to non-synapse membrane areas. Synaptic proteins may be involved in regulation of neurotransmitter release and reception, holding the synaptic structure and/or in the early development of neurons.

Synapses are the connection between neurons and are responsible for neuronal interaction. The synaptic function requires many proteins to hold the synapse together and transmit messages between the neurons. Synapses are changed in response to an activity called synaptic plasticity, which is considered responsible for learning and memory. Synaptic loss and dysfunction occur early, before cell death, in different neurodegenerative diseases (Lleo′ et al., 2019, Molecular & Cellular Proteomics, 18:546-560). Synaptic proteins have been suggested as biomarkers for neurodegenerative diseases and were measured mainly in CSF and plasma (Camporesi et al., 2020, Biomarker Insights, 15:1-17; Vrillon et al., 2022, Alzheimer's Research & Therapy, 14:71). Most attempts to measure synaptic proteins rely on immunoprecipitation followed by mass spectrometry. Developing antibody sets for membrane proteins like synaptic proteins is notoriously difficult, and reliable assays are unavailable.

The Luminex-compatible assay disclosed herein advantageously requires just one antibody against a synaptic protein being analyzed, rather than an antibody pair as required by traditional immunoassays. The detection is achieved by pairing the synaptic protein antibody with an antibody against a canonical EV marker. As exemplified herein below, the assay disclosed herein enables simultaneous detection, quantification and analysis of a plurality of synaptic proteins presented on the surface of EVs, using very small volumes of blood-derived samples. The assay disclosed herein is much more simple and accurate compared to hitherto described methods and therefore particularly suitable and useful for clinical diagnostic applications.

Synaptic proteins useful as targets for capture of EV according to the present invention include, for example, glutamate receptor subunit 2 (GluR2), neurogranin (NRGN), growth-associated protein 43 (GAP43), postsynaptic density 95 (PSD95) and Syntaxin-1 (STXN-1).

“Simultaneous” detection/analysis/quantification as disclosed herein means that the plurality of microsphere populations utilized by the methods of the present invention are subjected to fluorescence analysis and measurement in parallel. Typically, the plurality of microsphere populations are present in the same mixture and subjected to the fluorescence analysis and measurement in parallel.

“Tetraspanins”, also referred to as the transmembrane 4 superfamily (TM4SF) proteins, are membrane-spanning proteins with a conserved structure that function primarily as membrane protein organizers. Tetraspanins have four transmembrane alpha-helices and two extracellular domains, one short (called the small extracellular domain or loop, SED/SEL or EC1) and one longer, typically 100 amino acid residues (the large extracellular domain/loop, LED/LEL or EC2). Although several protein families have four transmembrane alpha-helices, tetraspanins are defined by conserved amino acid sequences including four or more cysteine residues in the EC2 domain, with two in a highly conserved ‘CCG’ motif. Extracellular vesicles are highly enriched in tetraspanins and some are characterized by a broad tissue distribution, thus serving as canonical markers of EVs. Examples include CD9, CD63 and CD81.

“Extracellular vesicles” (abbreviated EVs/EV) as used herein, refers to double membrane vesicle of various biogenesis pathways released into body fluids, and includes both vesicles released from living cells and from dying cells. The EVs captured and analyzed according to the present invention typically range in size between 10 nm to 200 nm (in diameter).

Analysis according to the present invention is preferably carried out using the Luminex platform, which employs a series of color-coded magnetic microspheres that are coated with target-specific capture antibodies. “Capture” of EVs refers to physical binding/association that following washing of unbound material results in separation of the EVs from other components of the plasma or serum sample in which they were present. Captured EV populations are then incubated with a set of detection antibodies, for example against canonical EV surface markers, in order to quantify the level of the targets that were captured by the capture antibodies. Detection antibodies are specific to a target on the surface of EV which is different from the target used for capture. For example, antibodies against a plurality of tetraspanins as disclosed herein may be used as detection antibodies. A detection antibody is conjugated to a label, such as phycoerythrin (PE) or to biotin+streptavidin-phycoerythrin (SAPE).

In some embodiments, captured EVs are further purified, for example, such that the EVs are at least 80%, at least 85%, at least 90%, at least 95% separated from other components of the plasma or serum sample. Each possibility represents a separate embodiment of the present invention. To obtain purified EVs, subsequent step(s) involving e.g. elution of the EV from the capture antibodies/beads under conditions involving e.g. elevated detergent content and/or reduced pH, is/are carried out.

In some embodiments, a plurality of magnetic microsphere populations appended with antibodies against tissue-specific surface markers that indicate the cellular origin of EVs are used to capture simultaneously a plurality of EV populations from a plasma or serum sample.

In some embodiments, a plurality of magnetic microsphere populations appended with antibodies against a plurality of synaptic proteins are used to capture simultaneously EVs that display at least one of the plurality of synaptic proteins on their surface.

A plurality of capture antibodies (namely, at least two capture antibodies) directed to distinct targets are used, wherein a first capture antibody is specific to a first target on the surface of EV, a second capture antibody is specific to a second target on the surface of EV, and so forth. The antibodies are conjugated or otherwise bound to magnetic microspheres, to form distinct populations of magnetic microspheres, each displaying antibodies directed to a distinct target on the surface of EV. For example, in some embodiments, a plurality of populations of magnetic microspheres are provided, each displaying antibodies directed to a distinct tissue-specific target on the surface of EV of a distinct cellular origin.

Providing a separate assessment of the level of the surface markers corresponding to each of the EV populations may be performed by generating gates encompassing the fluorescence range of each microsphere type corresponding to each distinct EV population and quantifying the fluorescence emission levels corresponding to the detection antibody for data points within each gate.

In another embodiment, said plurality of detection antibodies is directed to a plurality of tetraspanin markers. In another embodiment said tetraspanin markers are selected from the group consisting of CD9, CD63 and CD81. In another embodiment said plurality of detection antibodies comprises antibodies directed to CD9, antibodies directed to CD63 and antibodies directed to CD81. In another embodiment the fluorescent marker for labeling said plurality of detection antibodies comprises quantum dots or a combination of multiple fluorophores. Without wishing to be bound by a specific theory or mechanism of action, such combination of antibodies labeled by the same fluorescent marker provides for enhancement of the signal and the sensitivity of detection.

In another embodiment, said plurality of detection antibodies is directed to a plurality of diagnostic markers. In another embodiment, the plurality of detection antibodies is directed to surface EV markers that identify cell functions or subpopulations, examples include but are not limited to LAMP2, LC3, GAP43, annexins and integrins. In another embodiment, at least one labeled detection antibody is directed to a therapeutic target (also referred to herein as a disease-specific target) that can be found on EV surface, with the goal of quantitative or semi-quantitative assessment in different EV subpopulations. In a particular embodiment, the use of diagnostic markers comprising complement system components (e.g. C3, C5b-9, C4, Clq, C9, C3b, iC3b, TF, CRP, pCRP, MAC, CD59, CD55, CR1, C5aRl, or C5a) is explicitly excluded. In yet another embodiment, said marker is not an alpha-synuclein marker.

In another embodiment, the system comprises at least three and typically at least four populations of fluorescence-labeled magnetic microspheres. In another embodiment, each population of the distinct fluorescence-labeled magnetic microspheres comprises a distinct combination of fluorophores, enabling its discrimination from the other microsphere populations. In another embodiment said fluorescence-labeled magnetic microspheres are fluorescent magnetic microspheres compatible with Luminex detection devices (e.g., MagPlex microspheres).

In another embodiment, the targets of the capture system comprise a neural cell target and targets corresponding to at least three additional cellular origins selected from the group consisting of: bone, lung, tissue macrophage, lung macrophage, muscle, adipocyte, epithelium, endothelium, monocyte, microglia, megakaryocyte, T cell, erythrocyte, liver and oligodendrocyte. In another embodiment the targets of the capture system comprise a neural cell target and targets corresponding to at least three additional cellular origins selected from the group consisting of: bone, lung, tissue macrophage, lung macrophage, muscle, adipocyte, epithelium, endothelium, microglia, liver and oligodendrocyte. In another embodiment the targets of the capture system comprise a neural cell target and targets corresponding to at least three additional cellular origins selected from the group consisting of: bone, lung, tissue macrophage, lung macrophage, muscle, adipocyte, epithelium, endothelium, and microglia.

In another embodiment the targets of the capture system comprise GAP43 or CD171 and at least one additional target selected from the group consisting of P2RY12, CD68. In another embodiment said capture system comprises a population of magnetic microspheres displaying an antibody directed to GAP43, labeled by a first combination of fluorophores, a second population of magnetic microspheres displaying an antibody directed to CD68, labeled by a second combination of fluorophores, and a third population of magnetic microspheres displaying an antibody directed to P2RY12, labeled by a third combination of fluorophores. In another embodiment said capture system further comprises a fourth population of magnetic microspheres displaying an antibody directed to CD235a, labeled by a fourth combination of fluorophores. In another embodiment said targets further comprise at least one additional target as set forth in Table 4.

Advantageously, analytical methods in accordance with the invention provide for bypassing the enrichment and purification steps that require larger sample volumes. Thus, in another embodiment, the method does not include additional steps of EV isolation and/or sample processing, intended to enrich the biofluid sample with EV prior to incubation with the system. In some embodiments, a blood-derived sample analyzed according to the present invention is not subjected to a process to isolate or enrich the EV prior to incubating with the capture system disclosed herein. For example, the methods of the invention are herein demonstrated to provide high accuracy in multiplexed measurements of EV markers using unprocessed plasma samples of e.g., 50 μl plasma, without employing EV immunoprecipitation, size exclusion chromatography or similar steps that were required in hitherto reported assays. In yet other embodiments, e.g. when the isolation or specific capture of nano-EV is required, as will be discussed in further detail below, the method may include a step of size exclusion chromatography or the like.

Profiling of EV surface proteins may also serve as an important quality control measure in the production of therapeutic EV, by providing quality assurance and reproducibility of EV isolation. For diagnostic purposes, such profiling of EV surface proteome offers normalization criteria, which are currently highly lacking in the EV diagnostic space.

In certain embodiments, analytical methods of the invention may also be employed in therapy and diagnosis, for example in evaluating the circulating EV profiles of subjects in need thereof, or for quality control purposes, e.g. for elucidating EV origin or verifying the tissue selectivity of EV compositions intended for drug delivery or other therapeutic uses.

Thus, in another embodiment, analyzing the blood-derived sample comprises monitoring the tetraspanin profile of the sample (for example as a measure of monitoring the subject's health). In another embodiment analyzing the blood-derived sample comprises verifying a postulated cellular origin of an EV preparation. In another embodiment the EV preparation is for use in therapy. In another embodiment the postulated origin is neural, and a tetraspanin profile characterized by substantial similarity to the tetraspanin profile of a control neural EV population indicates that said preparation is of neural origin. In another embodiment the postulated origin is neural, and a tetraspanin profile characterized by enhanced surface levels of CD63 and CD81 compared to the surface levels of CD9 indicates that said preparation is of neural origin.

In some embodiments, methods according to the present invention comprise:
providing an unprocessed plasma or serum sample comprising extracellular vesicles (EVs);
providing a capture system comprising a plurality of populations of labeled microspheres, each microsphere population displaying antibodies directed to a distinct EV surface target and labeled with a distinct label;
incubating the plasma or serum sample with the capture system under conditions that allow specific binding between the antibodies and the EV surface targets while substantially maintaining the integrity of the EV membranes, to thereby provide distinct populations of EV-microsphere complexes corresponding to each target;
washing the EV-microsphere complexes under conditions enabling selective capturing of said complexes;
providing a plurality of detection antibodies directed to a plurality of EV surface markers, wherein the plurality of detection antibodies is labeled, directly or indirectly, with the same label;
incubating the captured EV-microsphere complexes with the plurality of detection antibodies under conditions that allow specific binding between the antibodies and EV surface markers while substantially maintaining the integrity of the EV membranes;
washing the resulting labeled complexes to remove excess reagents; and
subjecting the labeled complexes to a device amenable for simultaneously detecting and quantifying a plurality of signals, to quantify signal levels and provide a separate assessment of the level of the target protein corresponding to each of the EV populations.

In yet another aspect, there is provided a kit for analyzing blood-derived samples, comprising:
i) a capture system, comprising a first population of magnetic microspheres displaying an antibody directed to GAP43 or CD171, and labeled by a first combination of fluorophores, a second population of magnetic microspheres displaying an antibody directed to CD68, and labeled by a second combination of fluorophores, and a third population of magnetic microspheres displaying an antibody directed to P2RY12, and labeled by a third combination of fluorophores;
ii) a plurality of detection antibodies, each antibody directed to a distinct tetraspanin marker, wherein the plurality of detection antibodies is labeled, directly or indirectly, by the same fluorescent marker; and optionally
iii) reagents for performing said evaluation under conditions so as to retain said EV in a substantially intact form.

In another embodiment the fluorescent marker for labeling said plurality of detection antibodies comprises quantum dots or a combination of multiple fluorophores. In another embodiment, the reagents are selected from the group consisting of:
(i) at least one binding buffer for incubating a sample with the capture system to thereby provide distinct populations of EV-microsphere complexes, the at least one binding buffer characterized by lack of detergents and by the presence of protease and/or phosphatase inhibitors;
(ii) at least one washing buffer, characterized by significantly enhanced salt concentrations compared to the at least one binding buffer; and
(iii) at least one binding buffer and at least one washing buffer as defined in (i) and (ii) above.

According to additional embodiments, the invention relates to substantially purified populations of nano-EV, to processes for their preparation and methods for their use. As disclosed herein, nano-EV are distinguishable from EV obtained from the respective cultured cells in vitro. As further disclosed herein, nano-EV populations were identified as displaying markers of mature tissue-specific cells (e.g. neural markers), and are thus distinguishable from EV produced by stem cells or other pluripotent cells. Thus, nano-EV may be identified as tissue-specific EV. In addition, blood-derived EV, such EV displaying erythrocyte markers, were not included among the identified nano-EV populations, and were of a significantly larger size, corresponding to that of canonical EV. Accordingly, nano-EV may also be identified as non-blood-derived or as solid tissue-derived.

In another aspect, there is provided a process for isolating non-blood-derived tissue-specific EV from a biological sample, typically a blood-derived sample. In one embodiment, the process comprises a step of size selection (e.g. by filtration, centrifugation or chromatography-based methods) to thereby specifically select for EV that are smaller than 30 nm in diameter. In another embodiment, the selected EV are 10-25 nm in diameter (e.g. are 15-25 nm in diameter, 15-20 nm in diameter or 10-15 nm in diameter). In a particular embodiment, the size selection step comprises size exclusion chromatography. In another embodiment the step of size selection comprises size exclusion chromatography, nanoporous membrane filtration, deterministic lateral displacement sorting, dielectrophoretic isolation, acoustic fractionation, or combinations thereof. In another embodiment, the process further comprises isolating, purifying or analyzing the EV using affinity capture methods, e.g., as described herein above with respect to the analytical methods of the invention.

In one embodiment, the EV population is neuron-specific. In another embodiment, the EV population is macrophage specific. In another embodiment, said isolated EV comprise at least one EV population selected from the group consisting of neural-derived EV, and tissue macrophage-derived EV. In yet other embodiments, nano-EV populations may be isolated from blood-derived samples and identified using e.g., immuno-affinity capture methods based on the tissue-specific and/or tetraspanin profiles as disclosed herein. Each possibility represents a separate embodiment of the invention.

In another embodiment said isolated EV are characterized by a marker profile corresponding to differentiated solid tissue cells. In another embodiment the marker profile comprises protein markers, RNA markers, lipid markers or combinations thereof (e.g. markers as exemplified herein).

In some embodiments, an extracellular vesicle (EV) preparation is provided, comprising a substantially purified population of circulating neural-derived EV, characterized by a size distribution of 10-25 nanometers (nm) in diameter and a marker profile corresponding to neural cells.

In some embodiments, an extracellular vesicle (EV) preparation is provided, comprising a substantially purified population of circulating tissue macrophage-derived EV, characterized by a size distribution of 10-25 nanometers (nm) in diameter and a marker profile corresponding to tissue macrophages.

In another embodiment, the process further comprises loading said isolated EV with an exogenous cargo. In another embodiment the exogenous cargo is a therapeutic agent. In another embodiment said exogenous cargo is selected from the group consisting of: small molecule drugs, peptides, polypeptides, antibodies, oligonucleotides, nucleic acid constructs, and gene editing agents. In another embodiment the process further comprises modifying said isolated EV to display a targeting agent. In another embodiment the targeting agent is directed to a target selected from the group consisting of tissue-specific targets, cell-specific targets and disease-specific targets. In another embodiment said targeting agent is selected from the group consisting of antibodies, receptors, ligands, aptamers, and combinations thereof.

In another embodiment, there is provided an EV preparation comprising a substantially purified population of non-blood-derived, tissue-specific EV, the population characterized by a size distribution of 10-25 nm in diameter and by a marker profile corresponding to differentiated solid tissue cells.

In another embodiment, there is provided an EV preparation comprising a substantially pure EV population produced by the process. In another embodiment the preparation comprises a pharmaceutical-grade purity of said EV population. In another embodiment said preparation is formulated in the form of a pharmaceutical composition, further comprising a pharmaceutically acceptable carrier, excipient or diluent. In another embodiment, the isolated EV are further loaded with an exogenous cargo, intended for delivery into a target cell or tissue. In one embodiment, the cargo comprises a small molecule drug, antisense RNA, DNA, gene editing systems (e.g. CRISPR), peptides, proteins and/or antibodies. In another embodiment said cargo comprises a gene therapy agent. In another embodiment the isolated EV are further engineered or otherwise manipulated to display a targeting agent.

In another embodiment, EV preparation is for use in therapy. In another embodiment said EV preparation is for use in treating a neurological disorder, e.g. Alzheimer's disease, Parkinson's disease, multiple sclerosis, amyotrophic lateral sclerosis and others. In another embodiment said EV preparation is used for treating a liver disorder such as fibrosis and cirrhosis. Without wishing to be bound by a specific theory or mechanism of action, EV preparations comprising nano-EV in accordance with the invention exhibit improved therapeutic properties, including, but not limited to, enhanced tissue penetration, improved stability and/or enhanced efficacy.

In another embodiment, an EV population produced by the process as disclosed herein may be used in diagnosis. For example, without limitation, said EV population may be further analyzed for their biomarker content, e.g., disease-specific biomarkers, tissue-specific biomarkers and/or tetraspanin biomarkers, as disclosed herein. For example, without limitation, the biomarkers may include neurogranin (NRGN), Orexin (HTCR), SOX1 and OLIG2 Each possibility represents a separate embodiment of the invention.

In one aspect, there is provided a process for isolating at least one population of non-blood-derived, tissue-specific EV from a blood-derived sample, the process comprising a step of size selection for EV that are under 30 nanometers (nm) in diameter, and isolation of the selected EV

In one embodiment, the isolated EV are 10-25 nm in diameter. In another embodiment, said isolated EV are 15-25 nm in diameter, 15-20 nm in diameter or 10-15 nm in diameter. Each possibility represents a separate embodiment of the invention.

In another embodiment, the step of size selection comprises size exclusion chromatography, nanoporous membrane filtration, deterministic lateral displacement sorting, dielectrophoretic isolation, acoustic fractionation, or combinations thereof. In another embodiment, the process further comprises immunoaffinity purification and/or analysis the at least one EV population.

In another embodiment, said isolated EV are characterized by a marker profile corresponding to differentiated solid tissue cells. In another embodiment, the marker profile comprises protein markers, RNA markers, lipid markers or combinations thereof.

In another embodiment, said isolated EV comprise at least one EV population selected from the group consisting of neural-derived EV, tissue macrophage-derived EV, and kidney-derived EV. In another embodiment said isolated EV comprise at least one EV population selected from the group consisting of neural-derived EV, and tissue macrophage-derived EV. Each possibility represents a separate embodiment of the invention.

In another embodiment, said isolated EV display GAP43. In another embodiment, said isolated EV do not display CD235a. In another embodiment, said isolated EV display podocin. In yet another embodiment, said isolated EV does not display podocin. In another embodiment, said EV are characterized by higher CD63 levels and lower CD9 levels than canonical EV (e.g. erythrocyte-derived EV).

In some embodiments, the population of EVs is characterized by a membrane lipid composition according to Table A.

TABLE A
Lipid content of EVs (late SEC fractions)

% Total lipid
Late fractions

| Lipid species | Average | Std |
|---|---|---|
| Cholesterol | 31.17 | 1.001 |
| Phosphatidylethanolamine | 18.09 | 2.14 |
| Phosphatidylethanolamine esters | 16.15 | 1.43 |
| Phosphatidylcholine | 16.98 | 1.80 |
| Phosphatidylserine | 5.46 | 0.09 |
| Dihydrosphingomyelin | 0.56 | 0.01 |
| Sphingomyelin | 5.865 | 0.23 |
| Sulfatide | 0.425 | 0.05 |
| Ceramide | 0.59 | 0.02 |
| Phosphatidylinositol | 2.007 | 0.20 |
| Lactosyl-ceramide | 0.15 | 0.01 |
| Phospho-ceramide | 1.19 | 0.1 |
| Monohexosyl-ceramide | 0.28 | 0.01 |
| Lyso-phosphatidyl ethanolamine | 0.14 | 0.02 |
| Monoglycerides | 0.64 | 0.24 |
| Cholesterol esters | 0.26 | 0.04 |
| Lyso-phosphatidyl choline | 0.08 | 0.007 |
| Lyso-phosphatidylethanolamine | 0.06 | 0.01 |
| Diglycerides | 0.05 | 0.009 |

In another embodiment, the process further comprises loading said isolated EV with an exogenous cargo. In another embodiment, the exogenous cargo is a therapeutic agent. In another embodiment, said exogenous cargo is selected from the group consisting of: small peptides, polypeptides, antibodies, oligonucleotides, nucleic acid constructs, and gene editing agents. Each possibility represents a separate embodiment of the invention.

In another embodiment, the process further comprises modifying said isolated EV to display a targeting agent. In another embodiment, the targeting agent is directed to a target selected from the group consisting of tissue-specific targets, cell-specific targets and disease-specific targets. In another embodiment, said targeting agent is selected from the group consisting of antibodies, receptors, ligands, aptamers, and combinations thereof. Each possibility represents a separate embodiment of the invention.

In another aspect, there is provided an EV preparation comprising a substantially purified EV population isolated by the process as disclosed herein.

In another embodiment, the EV preparation comprises a pharmaceutical-grade purity of said at least one EV population. In another embodiment, said preparation is formulated in the form of a pharmaceutical composition, further comprising a pharmaceutically acceptable carrier, excipient or diluent.

In another embodiment, the EV preparation is for use in therapy. In another embodiment, the therapy comprises delivery of the exogenously loaded therapeutic agent to a target cell or tissue of a subject in need thereof. In yet another embodiment, the EV preparation is for use in diagnosis.

In another aspect, the EV preparation comprises a substantially purified population of non-blood-derived, tissue-specific EV, the population characterized by a size distribution of 10-25 nm in diameter and by a marker profile corresponding to differentiated solid tissue cells.

In another aspect, there is provided a method of analyzing a blood-derived sample, comprising:
a. providing a capture system, comprising at least four populations of distinct fluorescence-labeled magnetic microspheres, wherein each microsphere population displays antibodies directed to distinct targets on the surface of EV populations of distinct cellular origins,
b. providing a blood-derived sample of the subject, the sample comprising less than 75 μl of non-processed plasma, or a corresponding amount of intact EV;
c. incubating the sample with the capture system, under conditions such as to allow specific antigen-antibody binding while substantially maintaining the integrity of the EV membranes, to thereby provide distinct populations of EV-microsphere complexes corresponding to each target;
d. washing the EV-microsphere complexes using a magnetic device, under conditions enabling selective capturing of said complexes;
e. incubating said EV-microsphere complexes with a plurality of detection antibodies, each antibody directed to a distinct marker on the surface of the EV, under conditions such as to allow specific antigen-antibody binding while substantially maintaining the integrity of the EV membranes, wherein the plurality of detection antibodies is labeled by the same fluorescent marker;
f. washing the resulting labeled complexes using a magnetic device to remove excess reagents;
g. subjecting the resulting complexes to a microfluidic device amenable for detecting and simultaneously quantifying fluorescent emission on a plurality of wave lengths, to thereby quantify the fluorescence emission levels and provide a separate assessment of the level of the surface markers corresponding to each of the EV populations; and
h. comparing the quantified levels to control levels;
wherein the method is performed using reagents and under conditions so as to retain said EV in a substantially intact form.

In another embodiment, the fluorescent marker for labeling said plurality of detection antibodies comprises quantum dots or a combination of multiple fluorophores. In another embodiment, said plurality of detection antibodies is directed to a plurality of diagnostic markers. In another embodiment, said plurality of detection antibodies is directed to a plurality of tetraspanin markers. According to particular embodiments, said tetraspanin markers are selected from the group consisting of CD9, CD63 and CD81. In another particular embodiment, said plurality of detection antibodies comprises antibodies directed to CD9, antibodies directed to CD63 and antibodies directed to CD81.

In another embodiment, the targets of the capture system comprise a neural cell target and targets corresponding to at least three additional cellular origins selected from the group consisting of: bone, lung, tissue macrophage, lung macrophage, muscle, adipocyte, epithelium, endothelium, monocyte, microglia, megakaryocyte, T cell, erythrocyte, liver and oligodendrocyte. In another embodiment, the targets of the capture system comprise GAP43 and at least one additional target selected from the group consisting of P2RY12, CD68 and CD171. In another embodiment the targets of the capture system comprise GAP43 or CD171 and at least one additional target selected from the group consisting of P2RY12, CD68. In another embodiment, said capture system comprises a population of magnetic microspheres displaying an antibody directed to GAP43, labeled by a first combination of fluorophores, a second population of magnetic microspheres displaying an antibody directed to CD68 or CD171, labeled by a second combination of fluorophores, and a third population of magnetic microspheres displaying an antibody directed to P2RY12, labeled by a third combination of fluorophores. In another embodiment capture system comprises a population of magnetic microspheres displaying an antibody directed to GAP43, labeled by a first combination of fluorophores, a second population of magnetic microspheres displaying an antibody directed to CD68, labeled by a second combination of fluorophores, and a third population of magnetic microspheres displaying an antibody directed to P2RY12, labeled by a third combination of fluorophores.

In another embodiment, said capture system further comprises a fourth population of magnetic microspheres displaying an antibody directed to CD235a, labeled by a fourth combination of fluorophores. In another embodiment, said targets further comprise at least one additional target as set forth in Table 4 below. In another embodiment, said capture system comprises at least five, at least six, at least seven or at least eight populations of distinct fluorescence-labeled magnetic microspheres, wherein each microsphere population displays antibodies directed to distinct targets on the surface of EV populations of distinct cellular origins (e.g. at least five, at least six, at least seven or at least eight targets as set forth in Table 4).

In another embodiment, the sample is a plasma sample or a serum sample. In another embodiment, said sample comprises 1-50 μl of plasma or serum (e.g. 1-50 μl of unprocessed plasma or serum samples).

In another embodiment, analyzing the blood-derived sample comprises verifying a postulated cellular origin of an EV preparation. In another embodiment, the EV preparation is for use in therapy. In an exemplary embodiment, the postulated origin of the EV preparation is neural, and a tetraspanin profile characterized by substantial similarity to the tetraspanin profile of a control neural EV population indicates that said preparation is of neural origin. In another exemplary embodiment, the postulated origin is neural, and a tetraspanin profile characterized by enhanced surface levels of CD63 and CD81 compared to the surface levels of CD9 indicates that said preparation is of neural origin.

In another aspect, there is provided a kit for analyzing blood-derived samples, comprising:
i) a capture system, comprising a first population of magnetic microspheres displaying an antibody directed to GAP43, and labeled by a first combination of fluorophores, a second population of magnetic microspheres displaying an antibody directed to CD68 or CD171, labeled by a second combination of fluorophores, and a third population of magnetic microspheres displaying an antibody directed to P2RY12, labeled by a third combination of fluorophores;
ii) a plurality of detection antibodies, each antibody directed to a distinct tetraspanin marker, wherein the plurality of detection antibodies is labeled, directly or indirectly, by the same fluorescent marker; and optionally
iii) reagents for performing said evaluation under conditions so as to retain said EV in a substantially intact form.

In another embodiment, the capture system comprises a first population of magnetic microspheres displaying an antibody directed to GAP43 or CD171, and labeled by a first combination of fluorophores, a second population of magnetic microspheres displaying an antibody directed to CD68, labeled by a second combination of fluorophores, and a third population of magnetic microspheres displaying an antibody directed to P2RY12, labeled by a third combination of fluorophores.

In another embodiment, the fluorescent marker for labeling said plurality of detection antibodies comprises quantum dots or a combination of multiple fluorophores. In another embodiment, the reagents are selected from the group consisting of:
(i) at least one binding buffer for incubating a sample with the capture system to thereby provide distinct populations of EV-microsphere complexes, the at least one binding buffer characterized by lack of detergents and by the presence of protease and/or phosphatase inhibitors;
(ii) at least one washing buffer, characterized by significantly enhanced salt concentrations compared to the at least one binding buffer; and
(iii) at least one binding buffer and at least one washing buffer as defined in (i) and (ii) above.

These and other embodiments of the invention are described in further detail below.

### Antibodies

The invention in embodiments thereof relates to the use of binding reagents, including in particular antibodies. As used herein in the context of embodiments of the invention, the term antibody relates to at least an antigen-binding portion of an antibody.

An antibody directed (or specific) to an antigen, as used herein is an antibody which is capable of specifically binding the antigen. The term “specifically bind” or “specifically recognize” as used herein means that the binding of an antibody to an antigen is not competitively inhibited by the presence of non-related molecules.

Intact antibodies include, for example, polyclonal antibodies and monoclonal antibodies (mAbs). Exemplary functional antibody fragments comprising whole or essentially whole variable regions of both light and heavy chains (forming an antigen-binding portion) include, for example: (i) Fv, defined as a genetically engineered fragment consisting of the variable region of the light chain and the variable region of the heavy chain expressed as two chains; (ii) single-chain Fv (“scFv”), a genetically engineered single-chain molecule including the variable region of the light chain and the variable region of the heavy chain, linked by a suitable polypeptide linker; (iii) Fab, a fragment of an antibody molecule containing a monovalent antigen-binding portion of an antibody molecule, obtained by treating whole antibody with the enzyme papain to yield the intact light chain and the Fd fragment of the heavy chain, which consists of the variable and CH1 domains thereof; (iv) Fab′, a fragment of an antibody molecule containing a monovalent antigen-binding portion of an antibody molecule, obtained by treating whole antibody with the enzyme pepsin, followed by reduction (two Fab′ fragments are obtained per antibody molecule); and (v) F(ab′)2, a fragment of an antibody molecule containing a monovalent antigen-binding portion of an antibody molecule, obtained by treating whole antibody with the enzyme pepsin (i.e., a dimer of Fab′ fragments held together by two disulfide bonds). Further included within the scope of the invention are chimeric antibodies; recombinant and engineered antibodies, single-chained antibodies (e.g. single-chain Fv) and fragments thereof (comprises the antigen-binding portion).

The variable domains of each pair of light and heavy chains form the antigen binding site. The domains on the light and heavy chains have the same general structure and each domain comprises four framework regions, whose sequences are relatively conserved, joined by three hypervariable domains known as complementarity determining regions (CDR₁₋₃). These hypervariable domains contribute to the specificity and affinity of the antigen binding site.

The term “antigen” as used herein is a molecule or a portion of a molecule capable of being bound by an antibody. The antigen is typically capable of inducing an animal to produce antibody capable of binding to an epitope of that antigen. An antigen may have one or more epitopes. The specific reaction referred to above is meant to indicate that the antigen will react, in a highly selective manner, with its corresponding antibody and not with the multitude of other antibodies which may be evoked by other antigens.

Methods of generating monoclonal and polyclonal antibodies are well known in the art. Antibodies may be generated via any one of several known methods, which may employ induction of in vivo production of antibody molecules, screening of immunoglobulin libraries, or generation of monoclonal antibody molecules by continuous cell lines in culture. These include, but are not limited to, the hybridoma technique, the human B-cell hybridoma technique, and the Epstein-Barr virus (EBV)-hybridoma technique. Besides the conventional method of raising antibodies in vivo, antibodies can be generated in vitro using phage display technology, by methods well known in the art (e.g. Current Protocols in Immunology, Colligan et al (Eds.), John Wiley & Sons, Inc. (1992-2000), Chapter 17, Section 17.1).

The following examples are presented in order to more fully illustrate some embodiments of the invention. They should, in no way be construed, however, as limiting the broad scope of the invention.

### EXAMPLES

### Material and Methods

Preparation and characterization of intact exosome Luminex (IEL) beads: Antibodies were conjugated to functionalized magnetic microspheres in desired luminescence range, functionalized with carboxyl groups (MagPlexR, Luminex Corp., Cat. No. MC1XXXX-01), using 1-Ethyl-3-(3-dimethylaminopropyl) carbodiimide (EDC) chemistry (He et al. 2007, Bioconjug Chem 18:983-988). Bead recovery/concentration after conjugation was determined in a Countess™ 3 FL Automated Cell counter (Thermo Fisher Scientific, Cat. No. A49866) using reusable chamber slides (Thermo Fisher Scientific, Cat. No. A25750). The beads were stored in a blocking buffer containing 0.01-0.5% sodium azide and 0.5-5% bovine serum albumin (BSA) in PH between 7.5-9, at a minimum concentration of 1×10⁶/ml, up to three months.

To measure antibody loading, IEL beads were resuspended at 5.0*10⁶/ml in Assay Diluent containing 0.1-2% bovine serum albumin in phosphate buffered saline with proteases and phosphatase inhibitors and without a detergent, the PH of 7.5-8.5 and loaded in duplicates into in 96 well plates, black, (BrandTech., Cat. No. 781671), a minimum of 200 beads/well and an equal volume of appropriate serially diluted phycoerythrin (PE)-conjugated secondary antibodies was added. Assay Diluent only was used as a background control. The staining was carried out for at least 20 min at room temperature in a Genie® Microplate Mixer/Shaker (600 RPM), the beads were washed three times in a washing buffer containing PBS with TRIS 10-100 mM NaCl 100-500 mM and PH 7.5-8, resuspended in xMAP Sheath Fluid Plus (100 μl/well, Thermo Fisher Scientific, Cat. No. 4050021). The plates were analyzed in a Luminex200 plate reader, a minimum of 100 beads per condition.

Preparation of biotinylated detection antibodies: Antibodies were biotinylated by overnight incubation at −4° C. with EZ-link™ (Thermo Fisher Scientific,), at twenty-fold molar excess, per manufacturer's instructions. Excess reagent was removed using 7K MWCO Zeba™ Spin Desalting Columns, (Thermo Fisher Scientific, Cat. No. 89882).

IEL procedure: 50 μl of test sample diluted to desired concentration with Assay Diluent were placed in duplicates in the wells of a 96 well black plate. Assay diluent only was used as a background control. Working IEL beads/microspheres suspension in Assay Diluent was generated to yield 2.5-5.0×10³ beads/ml for each antibody/bead combination, for up to seven IEL analytes and 50 μl of working suspension added to each well. The pull-down was carried out overnight (16-18 hrs) at 4° C. on a Genie microplate shaker (400-1200 RPM), or in room temperature for 3 hours, or in 37° C. for 2 hours. The plates were washed three times in a washing buffer containing PBS with TRIS 10-100 mM NaCl 100-500 mM and PH 7.5-8 in a magnetic plate holder and the beads resuspended in Assay Diluent containing biotinylated detection antibody (1-2 μg/ml, 50 μl/well). After 1-4 hours incubation at room temperature (Genie shaker), the beads were washed three times, resuspended in 50 μl PBS containing Streptavidin-PE reagent (SAPE, 6 μg/ml), or streptavidin-Quantum dots incubated 20-60 min at room temperature, washed three times and resuspended in xMAP Sheath Fluid. The plate was analyzed on a Luminex200 reader as above.

Size exclusion chromatography: extracellular vesicles (EV) from the cell culture conditioned media were concentrated using 100 kDa MWCO Amicon concentrator (EMD Millipore, Cat. No. UFC910096), and plasma EV were concentrated by PEG-based precipitation. The concentrated samples (0.5 and 2 ml, as appropriate) were loaded on SEC columns (IZON qEV Original, 35 nm and IZON qEV2, 35 nm, for conditioned media and plasma, respectively). Void volume and up to 25 fractions (0.5 and 2 ml, respectively) were collected and protein concentrations (A280 absorption) were measured in each fraction, using NanoDrop2000 spectrophotometer (Thermo Fisher Scientific).

EV isolation from conditioned medium: conditioned medium (120 ml) was collected at BrainXell from induced pluripotent stem cells (iPSCs) differentiated into cortical neurons according to BrainXell protocol. HEK293 cells were grown with medium containing 10% FBS; upon reaching 50-70% confluence, the cells were washed extensively and were transferred into EV-free, serum-free basal medium and incubated for additional 48 hours. The medium was collected, cleared by two centrifugation rounds (10 min at 3,000 g) and EV were collected by ion exchange chromatography on DEAE Sephadex A-50 (20 mL) as described previously (Kosanovic et al. 2017, Biotechniques 63:65-71). The unbound material was washed with equilibration buffer (0.05 M Tris-HCl, pH 7.6), then weakly bound proteins and EV were eluted by step gradient with 0.25 and 0.5 M NaCl in 0.05 M Tris-HCl, pH 7.6. Finally, high-salt fraction containing EV was subjected to concentration and buffer exchange to PBS in a 100 kDa MWCO spin filter (Amicon)

Detergent treatment of crude EV fractions: Crude EV fractions generated by PEG-based precipitation (plasma EV) or by centrifugation/ultrafiltration through 100 kDa MWCO spin filter (Amicon) were supplemented with Triton X-100 to a final concentration of 2% and incubated 1-2 hours at room temperature or for 30 min at 50° C.

For RNA isolation, SEC fractions 2-5 and 8-13 were pooled and concentrated using 100 kDa MWCO Amicon filtration unit. RNA was isolated using miRNeasy serum/plasma kit (Qiagen, Cat. No 217184) following manufacturer's instructions, with modifications (treatment with RNAse free DNAse). cDNA was generated with SuperScript™ VILO™ cDNA Synthesis Kit (Cat. No 11754050) according to manufacturer's instruction. qPCR was performed using TaqMan kits and primers (Thermo Fisher Scientific, Cat. No. 44-445-56 and 4331182; for specific primers see Table 1 below).

TABLE 1
Primers used for EV analysis

| Gene transcript | Primer ID |
|---|---|
| Neurogranin (NRGN) | Hs00382922_m1 |
| SRY-Box Transcription Factor 1 (SOX1) | Hs01057642_s1 |
| Oligodendrocyte Transcription Factor 1 (OLIG1) | Hs00744293_s1 |
| Oligodendrocyte Transcription Factor 2 (OLIG2) | Hs00300164_s1 |
| Glyceraldehyde-3-Phosphate | Hs02786624_g1 |
| Dehydrogenase (GAPDH) | |
| Platelet Factor 4 (PF4) | Hs00427220_g1 |
| Heterozygous Beta-Globin/Hemoglobin (HBB) | Hs00758889_s1 |

Lipidomic analysis: Lipidomics profiling was performed by Ultra Performance Liquid Chromatography-Tandem Mass Spectrometry (UPLC-MSMS). Lipid extracts were prepared using a modified Bligh and Dyer method (Bligh and Dyer 1959), then spiked with appropriate internal standards, and analyzed using an Agilent 1260 Infinity HPLC integrated to Agilent 6490A QQQ mass spectrometer controlled by Masshunter V 7.0 (Agilent Technologies, Santa Clara, CA). Glycerophospholipids and sphingolipids were separated at 25° C. with normal-phase HPLC (Chan et al. 2012) using an Agilent Zorbax Rx-Sil column (2.1×100 mm, 1.8 μm) maintained as follows: mobile phase A (chloroform:methanol:ammonium hydroxide, 89.9:10:0.1, v/v) and mobile phase B (chloroform:methanol:water:ammonium hydroxide, 55:39:5.9:0.1, v/v); 95% A for 2 min, decreased linearly to 30% A over 18 min and further decreased to 25% A over 3 min, before returning to 95% over 2 min and held for 6 min. Separation of sterols and glycerolipids was carried out on a reverse phase Agilent Zorbax Eclipse XDB-C18 column (4.6×100 mm, 3.5 μm) using an isocratic mobile phase, chloroform, methanol, 0.1 M ammonium acetate (25:25:1) at a flow rate of 300 μl/min.

Quantification of lipid species was performed by multiple reaction monitoring transitions (Hsu et al. 2004; Guan et al. 2007; Chan et al. 2012) using both positive and negative ionization modes referenced to appropriate internal standards: PA 14:0/14:0, PC 14:0/14:0, PE 14:0/14:0, PG 15:0/15:0, PI 17:0/20:4, PS 14:0/14:0, BMP 14:0/14:0, APG 14:0/14:0, LPC 17:0, LPE 14:0, LPI 13:0, Cer d18:1/17:0, SM d18:1/12:0, dhSM d18:0/12:0, GalCer d18:1/12:0, GluCer d18:1/12:0, LacCer d18:1/12:0, D7-cholesterol, CE 17:0, MG 17:0, 4ME 16:0 diether DG, D5-TG 16:0/18:0/16:0 (Avanti Polar Lipids, Alabaster, AL). Lipid levels for each sample were calculated as a sum of the total molar amounts of all lipid species measured by all three LC-MS methodologies normalized to mol % and presented as mean mol %±S.E.M.

### Example 1. EV Capture and Detection Method Employing Luminex Technology Allows Specific Detection of Surface EV Proteins in Low Volume Plasma Samples

Antibody-conjugated beads for intact exosome Luminex (IEL) assays were generated as described above for the following surface antigens: CD9, a canonical EV marker, CD68, a macrophage marker, purinergic receptor P2RY12, a microglia marker, and neuronal marker axonal protein GAP43. These and other capture antibodies are listed in Table 2, below.

TABLE 2
Capture and detection (biotinylated)
antibodies for intact EV Luminex

| | | Cat. | | | Marker |
|---|---|---|---|---|---|
| Antigen | Vendor | No. | Host/Type | Specificity | specificity |
| CD9 | BioLegend | 312102 | Mouse | human | Pan-EV |
| | | | Monoclonal | | |
| CD63 | BioLegend | 353039 | Mouse | human | Pan-EV |
| | | | Monoclonal | | |
| CD81 | BioLegend | 349502 | Mouse | human | Pan-EV |
| | | | Monoclonal | | |
| CD171/ | Thermo | 14- | Mouse | human | Neuronal, |
| Biotin- | Fisher | 1719-82 | Monoclonal | | gut |
| CD171 | Scientific | | | | |
| CD235a/ | BioLegend | 349502 | Mouse | human | Erythroid |
| Biotin- | | | Monoclonal | | cells |
| CD235a | | | | | |
| GAP43/ | Thermo | MA5- | Rabbit | Human, | Neuronal |
| Biotin- | Fisher | 32256 | Monoclonal | mouse, rat | |
| GAP43 | Scientific | | | | |
| P2RY12 | BioLegend | 848002 | Rat | Mouse, | Microglia |
| | | | Monoclonal | human | |
| Biotin- | BioLegend | 312112 | Mouse | human | Pan-EV |
| CD9 | | | Monoclonal | | |
| Biotin- | BioLegend | 353018 | Mouse | human | Pan-EV |
| CD63 | | | Monoclonal | | |
| Biotin- | BioLegend | 349514 | Mouse | human | Pan-EV |
| CD81 | | | Monoclonal | | |

Beads were loaded using increasing concentrations of the antibodies (0.5, 1.0, 2.0 and 5.0 μg/10\hat{ }6 beads), with bead concentration not exceeding 5×10⁶/ml. Loading was assessed by staining with increasing concentrations of PE-conjugated secondary antibodies (as detailed in Table 3 below) and measuring in a Luminex200 reader (Thermo Fisher Scientific).

TABLE 3
Secondary antibodies

| Antibody | Vendor | Cat. No. |
|---|---|---|
| PE Goat anti-mouse IgG | BioLegend | 312102 |
| PE Donkey anti-rabbit IgG | BioLegend | 406421 |
| PE Goat anti-rat IgG | BioLegend | 405406 |

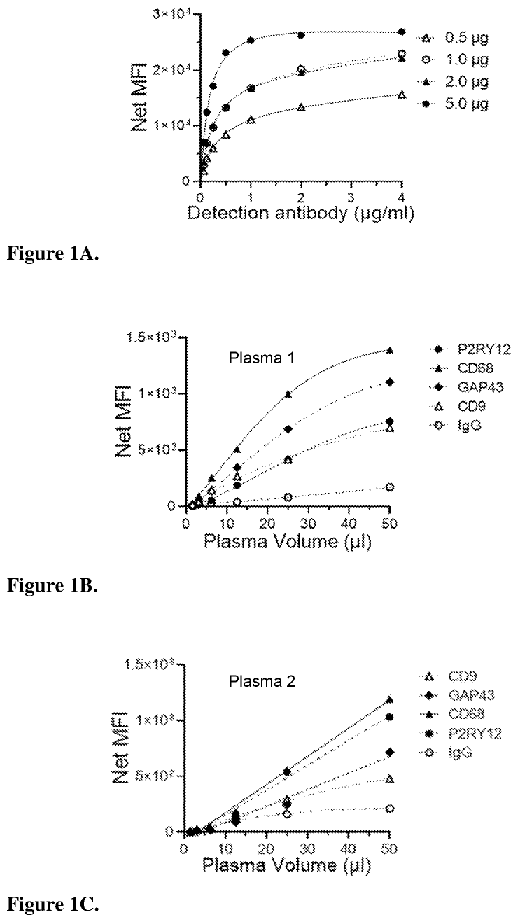

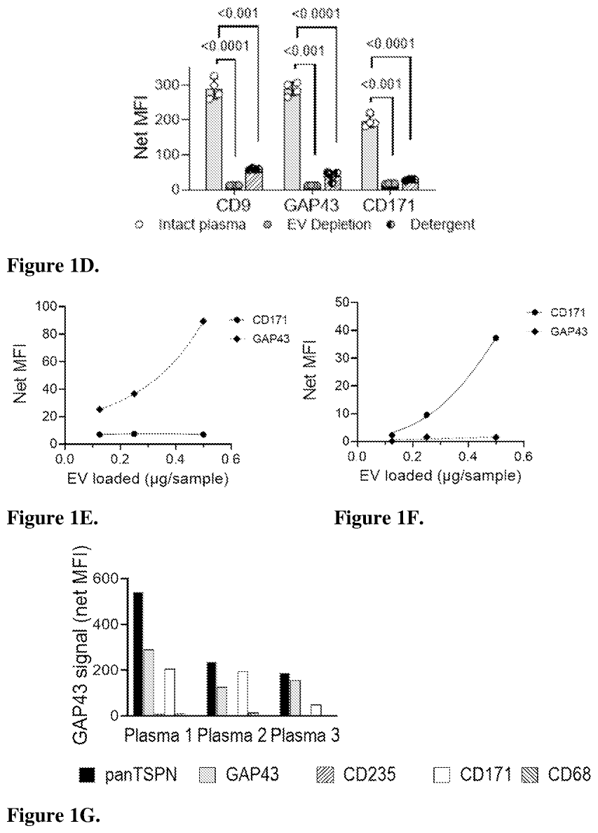

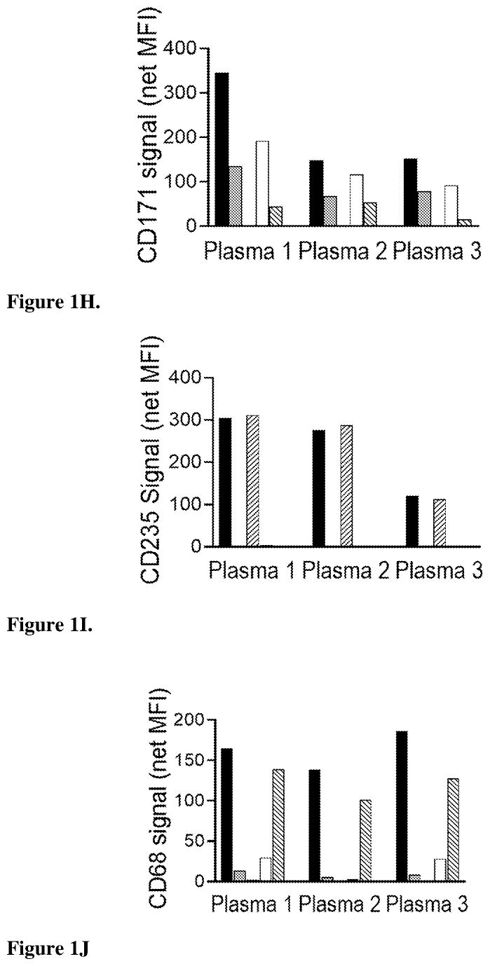

FIG. 1A shows anti-CD9 loading as an example. The figure exemplifies the net mean fluorescent intensity (MFI) obtained for loading the CD9 antibody on capture beads by using staining with increasing amounts of PE-conjugated anti-mouse antibodies (denoted “Detection antibody” in the figure), presented for the various loading concentrations (μg/10\hat{ }6 beads). For further bead production, antibody concentrations were chosen at the high end of the linear range where MFI is increased in proportion to the antibody concentration, but below saturation level (1 μg/10\hat{ }6 beads for the CD9 antibody and between 1 and 5 μg/10\hat{ }6 beads for other antibodies).

Beads generated according to this protocol were tested for EV capture in unprocessed, serially diluted plasma samples from healthy donors (BioIVT). The detection was carried out using a cocktail of biotinylated detection antibodies directed to canonical EV markers—the tetraspanin surface antigens CD9, CD63, and CD81 (thereafter referred to as panTSPN, see Table 2 for detection antibodies). The analysis of two random unprocessed plasma samples using beads conjugated with antibodies against specific targets showed strong dose-dependent signals with a linear assay range between 10 and 50 μl plasma input (1/5-1/1 plasma dilutions, FIGS. 1B, 1C). In contrast, beads appended with a non-specific IgG (a negative control) generated much lower signal, likely due to non-specific binding (FIGS. 1B, 1C). To further assess assay specificity, EV depletion using PEG-based precipitation was performed prior to analysis (essentially as described in Rider et al., Sci Rep 6, 23978 (2016)), or detergent lysis (Triton-X100, 2% final concentration). As shown in FIG. 1D, in both cases the signal was significantly reduced, demonstrating that the signal can be attributed specifically to the plasma EV.

Finally, medium conditioned by HEK293 cells overexpressing neuronal antigens, GAP43 or CD171 (see methods), was analyzed as described above, with the beads appended with either anti-CD171 or anti-GAP43 antibodies (FIGS. 1E and 1F). As can be seen in FIG. 1E, EV from the medium conditioned by the GAP43 overexpressing cells were detected with GAP43-specific and not with CD171-specific beads. Conversely, CD171-positive EV were detected by IEL beads appended with CD171 but not with GAP43 antibodies (FIG. 1F), thus revealing the specificity of detection. Together, these results demonstrate that the newly developed method detects a signal that is both EV-specific and antigen specific, in remarkably small volumes of unprocessed human plasma or conditioned media.

### Example 2. Specific EV Pulldown Results in Minimal Cross-Population Contamination

Next, EV of different cellular origins were captured using IEL beads conjugated with appropriate capture antibodies and detected using the panTSPN cocktail or with specific detection antibodies against GAP43, CD171, CD68 and CD235 (for specific antibodies see Table 2), essentially as described in Example 1. The results are shown in FIGS. 1G-1J. As can be seen in FIG. 1G, EV captured with IEL beads appended with antibodies against the neuronal marker GAP43 generated a strong positive signal when detected using panTSPN cocktail, GAP43 antibody and an antibody against another neuronal marker, CD171, but not with an antibody against macrophage (CD68) and erythrocyte (CD235) markers. Similarly, EV captured with CD171 IEL beads were detectable by panTSPN cocktail, GAP43 antibody and CD171 antibody. Modest positivity with CD68 detection is consistent with low-level CD171 expression by monocytes (FIG. 1H). Conversely, EV population generated by the pulldown with CD235 IEL beads was only detectable with panTSPN and CD235 antibodies and lacked positivity for GAP43, CD171 and CD68 (FIG. 1I). Finally, CD68-positive EV were detectable with a strong signal using panTSPN cocktail and CD68 antibody, and to a lesser degree using CD171 antibody (FIG. 1J). Thus, the results demonstrate remarkable specificity in capturing and detecting specific EV populations, including erythrocyte-derived EV and neuron-derived EV, characterized by minimal cross-reactivity with antigens specific for unrelated cell types.

### Example 3. Multiplexed Measurements of EV Surface Proteins

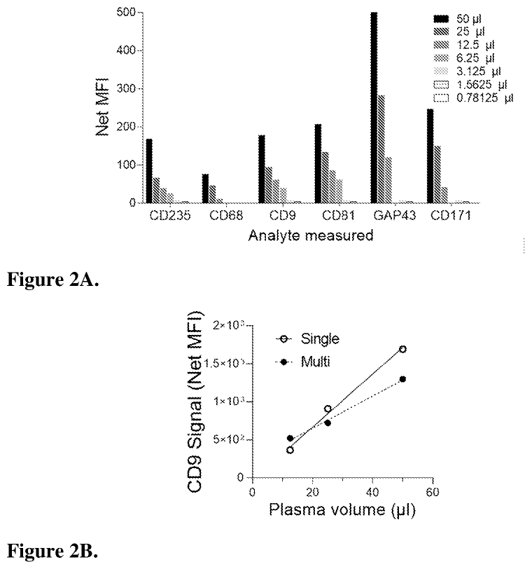

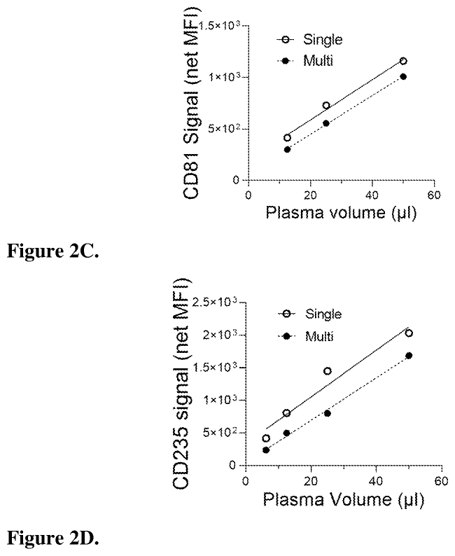

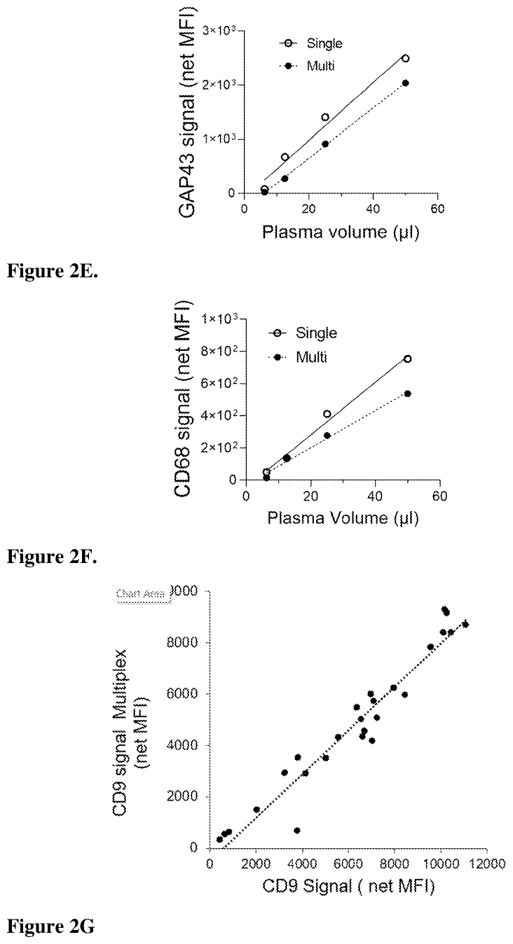

One of the biggest advantages of the Luminex platform is its multiplexing capacity. The sensitivity of multiplexed and single-bead IEL assays were compared for multiple tissue-specific and canonical EV surface markers, including the erythrocyte marker CD235 (Glycophorin A), the macrophage marker CD68, EV markers CD9 and CD81, and neuronal markers GAP43 and CD171. The results are presented in FIGS. 2A-2G. Capture was carried out using Luminex beads with the indicated antibodies, and the detection was performed using panTSPN. As can be seen in FIG. 2A, measurements in serial plasma dilutions showed a dose-dependent signal reduction for each analyte with a clear linear range between 10-50 μl. To assess the potential reduction in sensitivity due to multiplexing, dose/response curves were generated for both single-bead and multiplexed assays for each analyte. As can be seen in FIGS. 2B-2F, both types of assays generated similar curves within their linear range. As can be seen in FIG. 2G, measurements of CD9 in 25 random plasma samples produced significant differences between individual donors, but a strong correlation between single-bead and multiplexed assays. Thus, the results demonstrate the utility of the newly developed method for multiplexed measurements.

The assay was further tested and confirmed to be useful for multiplexed capturing of EV of various additional tissue origins. The details of the targets for the various capture antibodies used are summarized in Table 4 below.

TABLE 4
Targets for EV capture

| Cell type | Surface targets |
|---|---|
| Bone | CALCR, OSCAR, CD51, FGFR3 |
| Lung | SFTPC, SFTPA, SFTPB, CD44v6, |
| | HTII-280, HTI-56 |
| Lung macrophage | MRC1, MCEMP1 |
| Tissue macrophage | CD68 |
| Muscle | RYR1 |
| Adipocyte | PLIN1, HAS1 |
| Epithelium | CD66, MUC-1, MUC-4, MUC-6, nectin-1, |
| | CD166, CD140, AQP5, PDPN, |
| Neuron | GAP43, CD171 |
| Endothelium | CD146, CD38 |
| Monocyte | CD68, CD11B, IBA1 |
| Microglia | P2RY12, TMEDM119 |
| Megakaryocyte, | CD62P, CD31 |
| endothelium | |
| T-cell | CD3 |
| Erythrocyte | CD235a |
| Liver | SLC2A2 |
| Oligodendrocyte | PLP, OMG |

### Example 4. Exosomes of Different Cellular Origins Present with Unique Characteristic Tetraspanin Profiles

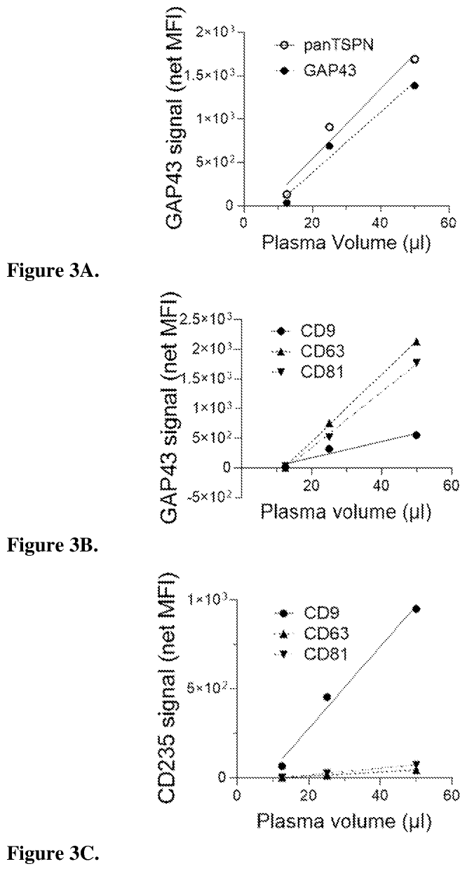

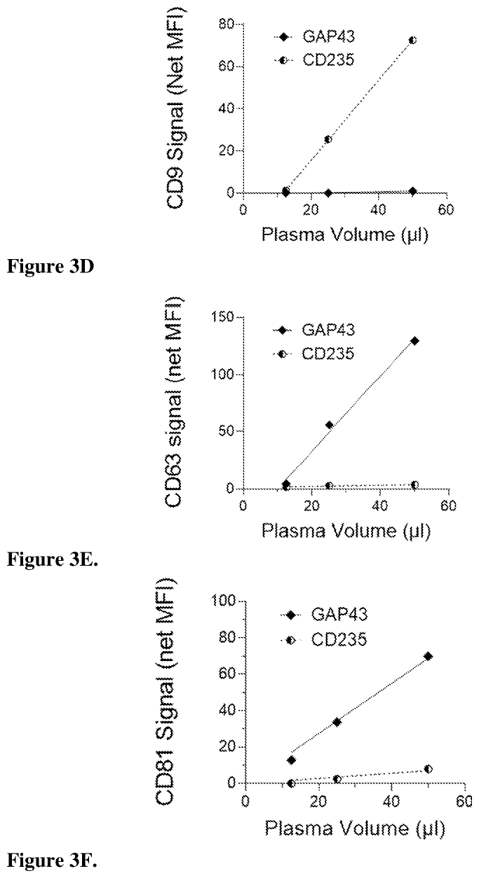

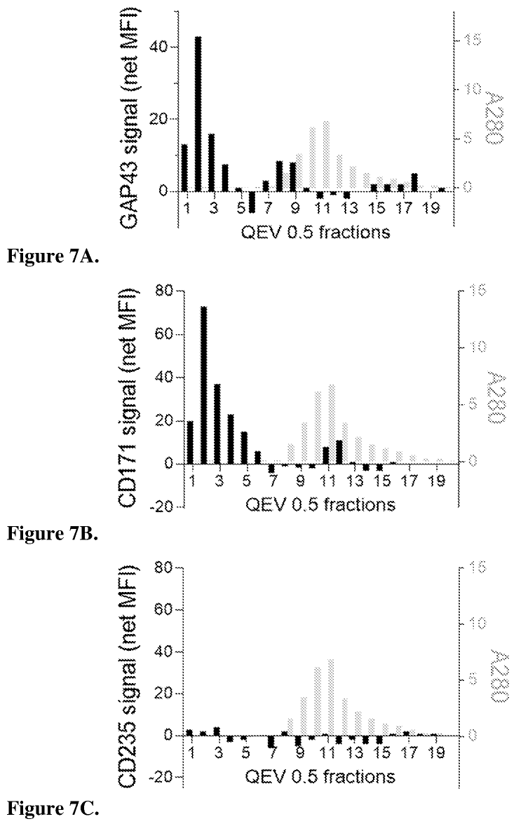

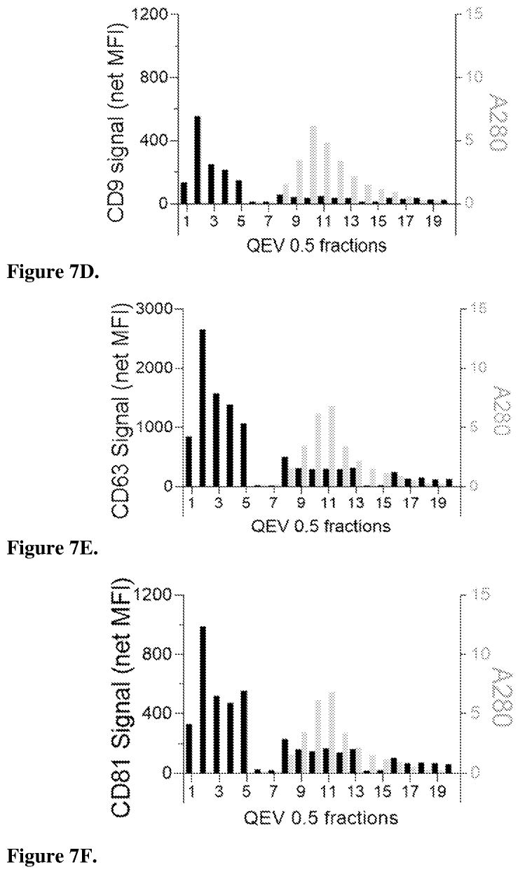

Next, multiple combinations of IEL capture beads with detection antibodies were used to examine potential differences in the expression of distinct surface proteins by EV of various cellular origins. The results are presented in FIGS. 3A-3F. As can be seen in FIG. 3A, when GAP43 (neuron-specific) IEL beads were used in combination with either a panTSPN antibody cocktail or with GAP43 antibody for detection, similar signal strength was recovered, indicating that most GAP43 is EV-associated. When GAP43 IEL beads were applied with either CD9, CD63, or CD81 detection antibodies, the signal generated with CD9 antibodies was significantly weaker (FIG. 3B), suggesting that CD63 and CD81 are the two major tetraspanins expressed by GAP43-positive neuronal EV. Interestingly, lower CD9 signal was also generated by neuronal cell culture-derived EV compared to the signal for CD63 and CD81, but not by HEK293 cells (FIGS. 7D-7F). When using CD235 IEL beads to capture erythrocytic EV, only CD9, but not CD63 or CD81 detection antibodies generated strong dose-dependent signal (FIG. 3C), suggesting that the majority of CD235-positive EV contain low levels of CD63 and CD81, but high levels of CD9. This correlation between surface marker and EV type suggests that differences in IEL signals may not be attributed to differences in antibody affinity, but to differential surface presentation of specific antigens by different EV types. Further, EV captured with CD9 IEL beads were positive for CD235 but not for GAP43 (FIG. 3D), and EV positive for CD63 or CD81 contained no detectable CD235 but produced a weak signal with GAP43 detection antibody (FIGS. 3E, F). These results demonstrate that the methods as disclosed herein are useful to characterize EV surface protein composition, assess the colocalization of markers on the EV surface and potentially identify EV subpopulations. Indeed, the data presented herein unexpectedly reveal the diverse and unique characteristic profiles of EV according to their cellular origin, a valuable tool in utilizing EV as a diagnostic measure.

### Example 5. Intact EV Luminex Combined with Size Exclusion Chromatography Uncovers Cell-Specific Variability of EV Sizes

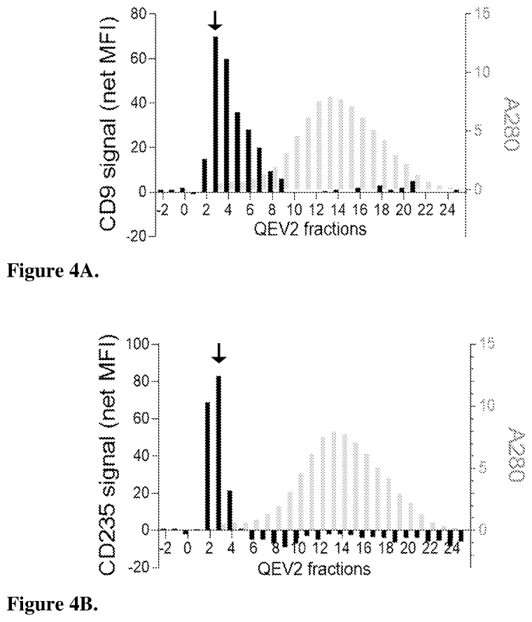

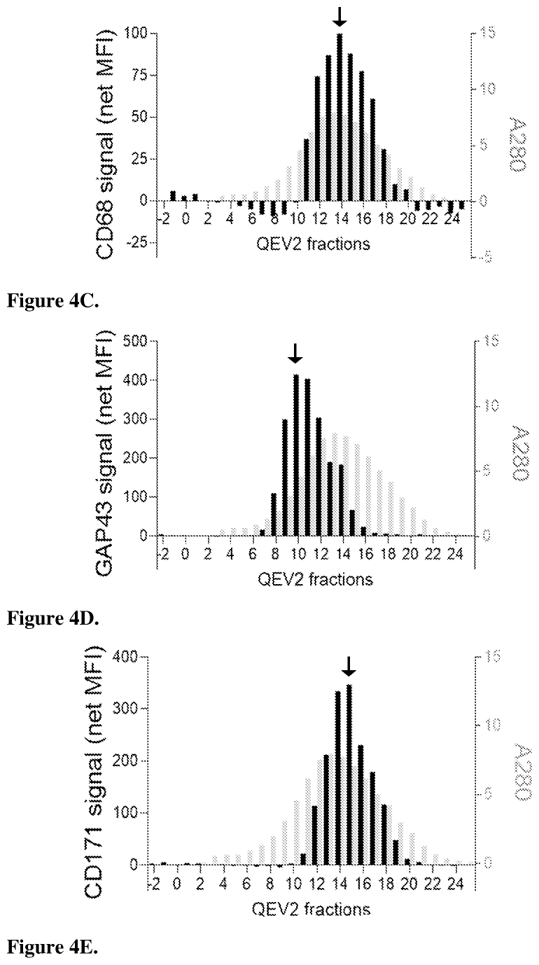

Next, size exclusion chromatography (SEC) of plasma EV was performed, and the fractions collected were subjected to the IEL capture and detection analyses essentially as described in Example 4. To ensure that signal is generated by the EVs, only the panTSPN cocktail was initially used for detection. To this end, plasma EV were concentrated by PEG precipitation (as described in the Methods section above), resuspended in PBS, and loaded on qEV2 columns (IZON Sciences). Several void fractions (negative numbers) and all elution fractions were analyzed by IEL with the beads appended with antibodies against CD9, CD235, CD68, GAP43 and CD171. The results are presented in FIGS. 4A-4E, elucidating the size range of EV detected by the assay.

As can be seen in FIG. 4A, the combination of CD9 IEL beads with the panTSPN detection cocktail produced the expected peak at fraction 3, trailing to fraction 9. CD235-positive EV detected with the panTSPN cocktail also peaked at fraction 3 (FIG. 4B). Thus, CD9-positive and CD235-positive EV collect in early fractions, consistent with the average diameter of >100 nm, e.g. 150-200 nm. Unexpectedly, IEL beads specific for CD68, a marker of tissue macrophages, and for the neuronal markers GAP43 and CD171, while detectable with the same panTSPN cocktail, produced strong signals in fractions 11-20, 8-15 and 11-20, respectively (FIGS. 4C-4E), indicative of smaller particle sizes of <30-50 nm, depending on the fraction.

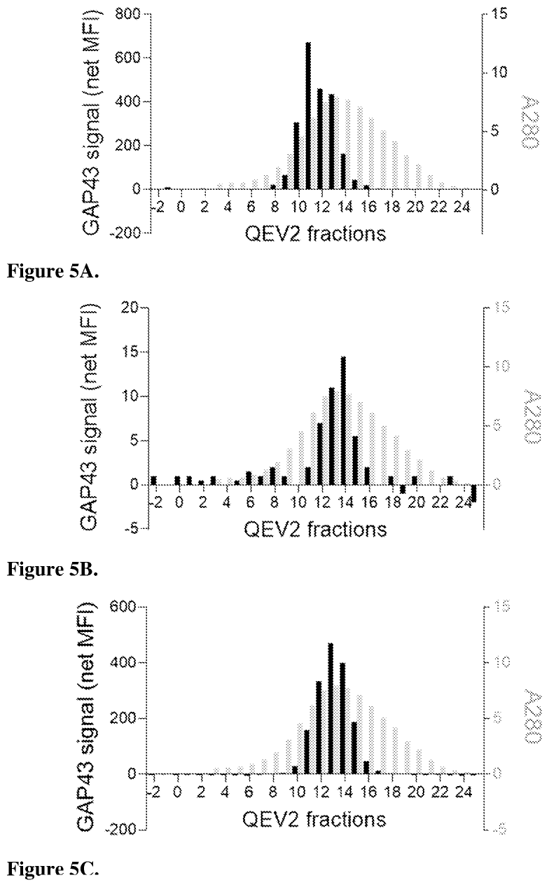

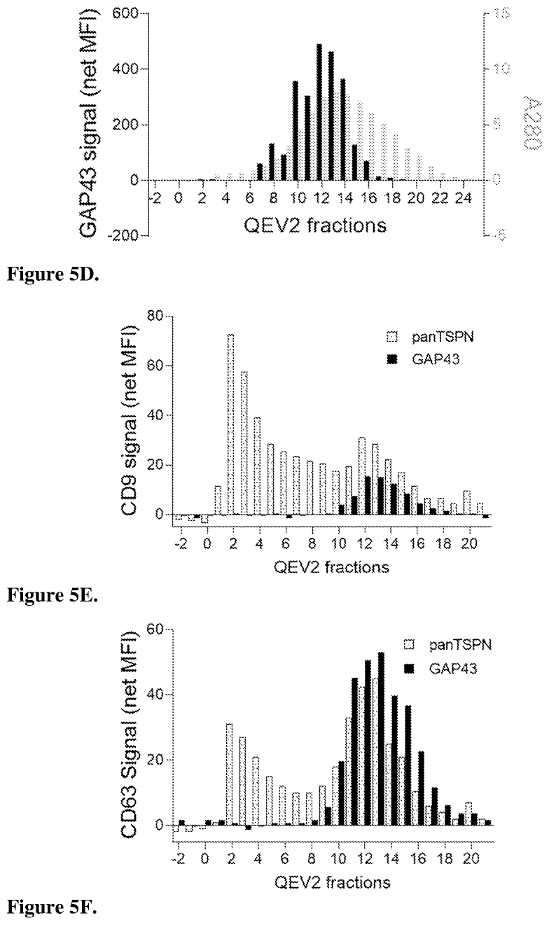

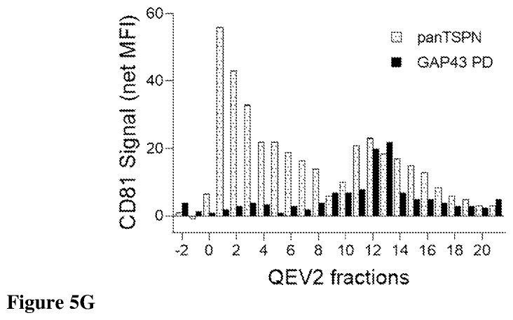

To further characterize the origin of these late fraction signals, the analysis was repeated, using GAP43 IEL beads followed by detection with panTSPN antibody cocktail or with specific TSPN antibodies (FIGS. 5A-5D). Interestingly, while panTSPN antibody cocktail yielded a strong positive signal in fractions 7-15, (FIG. 5A), the detection of CD9 alone resulted in a weak (<50 net MFI) positive signal in fractions 11-16 (FIG. 5B). CD63 and CD81 detection both yielded much stronger signals in fractions 9-15 and 7-16, respectively (FIGS. 5C, 5D). In agreement, CD9 IEL beads in combination with GAP43 detection antibody produced a weak signal in fractions 10-16, while CD63 and CD81 IEL beads combined with GAP43 detection antibody yielded signal in fractions above 10 (FIGS. 5E-5G).

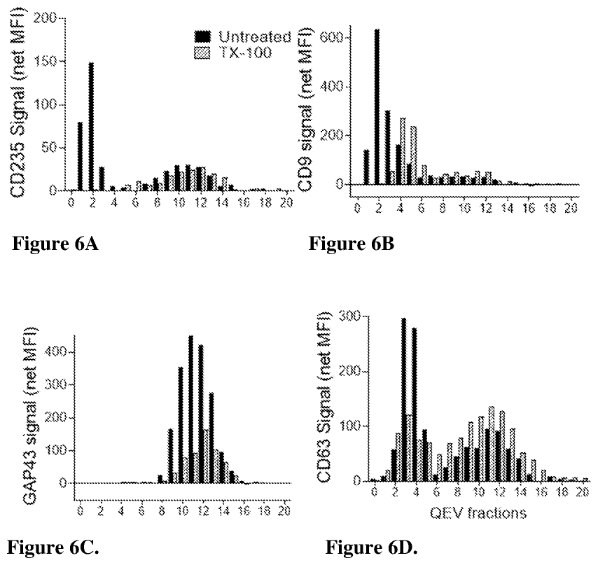

To ascertain whether the late-fraction signals detectable with pan-TSPN antibodies can be attributed to EV, fractionation of the crude EV preparations were subjected to detergent treatment to disrupt the associations between the EV and cell type-specific antigens via plasma membrane, in accordance with MISEV guidelines (Welsh et al. 2020, J Extracell Vesicles 9:1713526). Treatment with Triton-X100 at 50° C. resulted in significant reduction of the signal, both for early, CD9 and CD235-positive (FIGS. 6A, 6B), and for late, CD63 and GAP43-positive fractions (FIGS. 6C, 6D). Interestingly, pre-treatment with the same TX-100 concentration at room temperature resulted in diminished signal in the early, but not in the late fractions: this relative resistance to the TX-100 was indicative of increased lipid raft content within smaller EV membranes (see Example 6 below for lipid content analysis). Interestingly, IEL beads for CD63 and CD81, in addition to strong peaks in early fractions, generated positive signals dispersed towards later fractions (FIGS. 5F-G). In summary, the results presented herein demonstrate that plasma-derived EV of different cellular origins are surprisingly characterized by markedly distinct size distribution profiles, with EV of neuronal and macrophage origin exhibiting substantially smaller average particle sizes. Accordingly, the capture and analysis methods disclosed herein unexpectedly enable elucidation of cell-specific variability in EV sizes and their varied characteristic profiles, a unique attribute applicable to their diagnostic and therapeutic utility.

### Example 6. SEC Late Fractions Contain mRNA and Lipids Typical of EV

Previously it been postulated that the late SEC fractions contain soluble proteins and not membranous vesicles. Therefore, experiments were undertaken to examine whether the IEL signal detected in the late fractions was EV specific. To this end, RNA and lipid analysis of pooled fractions 2-5 and 8-13 were performed.

Cell-free mRNA is found in plasma in association with HDL, protein particles and EV, of which EV are considered to be the main mRNA carrier. To have enough material for quantitative analysis, early fractions (2-5) and late fractions (8-13) were combined and concentrated using 100 kDa MWCO Amicon spin filters prior to RNA extraction. The level of multiple cell-specific mRNA was measured by qPCR with TaqMan primers (Table 1 above). The results are shown in Tables 5-7 below, in which the values are presented as qPCR cycle threshold (Ct) and/or by the differences in cycle threshold (ΔCt) compared to a control transcript (GAPDH, HBB or PF4, respectively).

An observed ˜2-fold decrease in GAPDH content from early to late fractions (Ct values of 26.33 and 27.34, respectively) is suggestive of higher overall RNA level in early fractions, which is consistent with the predicted larger EV size and the absence of soluble protein; however, the clearly detectable mRNA levels in late fractions (see Tables 5-7 below) are indicative of EV presence. In addition, normalization to the housekeeping transcript (GAPDH), revealed 2-4-fold enrichment in neuron-specific transcripts, such as neurogranin (NRGN) and Orexin (HTCR) as well as the oligodendrocyte markers SOX1 and OLIG2, as indicated by late/early ΔCt ratio (2^{ΔCt early-ΔCt late}). The data for NRGN, SOX1 and OLIG2 is shown in Table 5 below.

TABLE 5
mRNA enrichment in pooled SEC
fractions (normalized to GAPDH)

| | ΔCt (to GAPDH) | | |
|---|---|---|---|
| Gene transcript | Early fractions | Late fractions | Late/early ratio |
| NRGN | 2.362234 | 0.407699 | 3.875911 |
| SOX1 | 2.589385033 | 0.71876812 | 3.656889 |
| OLIG1 | 2.192837715 | 1.758674622 | 1.351127 |
| OLIG2 | −2.35433 | −3.76834 | 2.664775 |

Taken together with high content of neuron-specific protein markers, e.g., GAP43 and CD171 in late SEC fractions, as was detected by IEL (see above), the observed enrichment of neuron-specific mRNA is indicative of distinct cellular origins of the EV in in the late fractions. In addition, the ratio between neuron-specific mRNA (NRGN) to mRNA encoding erythrocytic protein hemoglobin beta (HBB) is significantly higher in the later fractions (Table 6), further suggesting that in plasma, the majority of EV of neuronal origin are in the smaller size range.

TABLE 6
Neuronal and erythrocytic mRNA content in pooled SEC fractions

| | Ct | | ΔCT (to HBB) | | Ratio to HBB transcript | |
|---|---|---|---|---|---|---|
| Gene | Early | Late | Early | Late | Early | Late |
| transcript | fractions | fractions | fractions | fractions | fractions | fractions |
| NRGN | 22.99 | 23.05 | −5.61 | −8.06 | 48.93 | 267.63 |
| SOX1 | 28.92 | 28.06 | 0.31 | −3.04 | 0.80 | 8.28 |
| OLIG1 | 28.52 | 29.1 | −0.081 | −2.009 | 1.06 | 4.026 |
| OLIG2 | 23.97 | 23.57 | −4.62 | −7.53 | 24.72 | 185.64 |
| HBB | 28.60 | 31.11 | — | — | — | — |

Similar results were obtained when the ratio between neuron-specific mRNAs to platelet mRNA was determined (platelet factor 4, PF4, Table 7, below). PF4 mRNA is also found predominantly in early fractions and the ratio between NRGN and PF4 mRNA is much higher in late fractions, suggesting strong enrichment for neuronal transcripts

TABLE 7
Neuronal and platelet mRNA content in pooled SEC fractions

| | Ct | | ΔCT (to PF4) | | Ratio to PF4 transcript | |
|---|---|---|---|---|---|---|
| Gene | Early | Late | Early | Late | Early | Late |
| transcript | fractions | fractions | fractions | fractions | fractions | fractions |
| NRGN | 22.1 | 23.04 | −9.075 | −13.41 | 539.31 | 10900.63 |
| SOX1 | 28.92 | 28.0 | −3.14 | −8.4 | 8.85 | 337.16 |
| OLIG1 | 28.52 | 29.1 | −3.54 | −7.36 | 11.65 | 163.98 |
| OLIG2 | 23.97 | 23.57 | −8.09 | −12.88 | 272.5 | 7561.25 |
| PF4 | 32.07 | 36.46 | — | — | — | — |

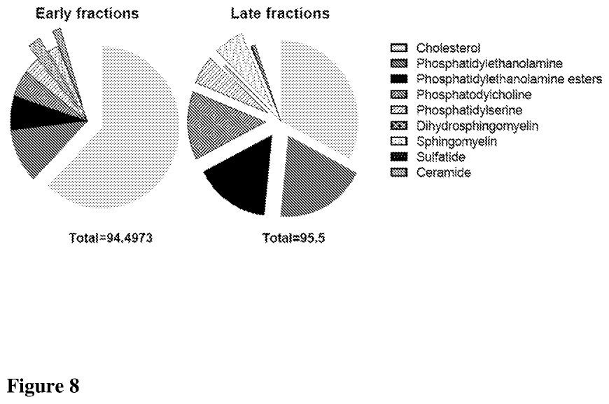

To further ascertain the EV presence in the late SEC fractions, the pooled, concentrated fractions 2-5 and 8-13 were subjected to lipidomic analysis (see methods). The lipid composition of the late fractions was consistent with the published data generated by the lipidomic analysis of EV membranes (Skotland et al. 2020, Adv Drug Deliv Rev 159:308-321), wherein for the major classes of lipids (cholesterol, sphingomyelin, phosphatidylcholine and PC ethers, phosphatidylserine, phosphatidylethanolamine, PE ethers, diglycerides, phosphoglycerates, phosphatidic acid, phosphatidyl inositol, ceramide and lactoceramide) the measured percentage of total lipid content in the late and early EV fractions was within the previously observed range. Interestingly, early, and late SEC fractions also presented with several significant differences in the lipid content (Table 8 below and FIG. 8).

TABLE 8
Lipid content of the late and early pooled SEC fractions

| | % Total lipid | | | | Late/ | |
|---|---|---|---|---|---|---|
| | Early fractions | | Late fractions | | Early | P value |
| Lipid species | Average | Std | Average | Std | ratio | (T-test) |
| Cholesterol | 58.84 | 12.78 | 31.17 | 1.001 | 0.52 | 0.01 |
| Phosphatidylethanolamine | 7.18 | 1.79 | 18.09 | 2.14 | 2.51 | 0.006 |
| Phosphatidylethanolamine esters | 6.55 | 1.63 | 16.15 | 1.43 | 2.46 | 0.01 |
| Phosphatidylcholine | 5.065 | 3.5 | 16.98 | 1.80 | 3.35 | 0.001 |
| Phosphatidylserine | 2.804 | 0.03 | 5.46 | 0.09 | 1.94 | 3.71E−05 |
| Dihydrosphingomyelin | 2.66 | 0.42 | 0.56 | 0.01 | 0.21 | 0.044 |
| Sphingomyelin | 2.067 | 0.11 | 5.865 | 0.23 | 2.83 | 0.0002 |
| Sulfatide | 1.043 | 0.12 | 0.425 | 0.05 | 0.40 | 0.03 |
| Ceramide | 1.01 | 0.10 | 0.59 | 0.02 | 0.58 | 0.04 |
| Phosphatidylinositol | 1.001 | 0.11 | 2.007 | 0.20 | 1.98 | 0.003 |
| Lactosyl-ceramide | 0.8 | 0.20 | 0.15 | 0.01 | 0.19 | 0.06 |
| Phospho-ceramide | 0.34 | 0.03 | 1.19 | 0.1 | 3.45 | 0.001 |
| Monohexosyl-ceramide | 0.33 | 0.01 | 0.28 | 0.01 | 0.85 | 0.03 |
| Lyso-phosphatidyl ethanolamine | 0.22 | 0.02 | 0.14 | 0.02 | 0.63 | 0.04 |
| Monoglycerides | 0.17 | 0.004 | 0.64 | 0.24 | 3.61 | 0.03 |
| Cholesterol esters | 0.17 | 0.009 | 0.26 | 0.04 | 1.49 | 0.03 |
| Lyso-phosphatidyl choline | 0.15 | 0.015 | 0.08 | 0.007 | 0.56 | 0.038 |
| Lyso-phosphatidylethanolamine | 0.14 | 0.02 | 0.06 | 0.01 | 0.47 | 0.03 |
| Diglycerides | 0.12 | 0.04 | 0.05 | 0.009 | 0.46 | 0.13 |

First, free cholesterol content in the late fractions was approximately 2 times lower compared to the early fractions, suggesting that part of cholesterol in these fractions could be sequestered in protein complexes involved in its transport in circulation. This was consistent with 2-3-fold increases in the amounts of phosphatidylethanolamine (PE), PE esters, phosphatidylcholine, and phosphatidylserine (Table 8). On the other hand, significant 2.5-fold increase in sphingomyelin (SM) content and increased SM/cholesterol ratio, point to higher proportion of lipid raft domains. Together with relative resistance to Triton-X100 (the TSPN-positive signal in late fractions was diminished by after 30 min TX-100 treatment of crude plasma EV preparation at 50° C., but not at room temperature, as was reported above), these observations suggest higher lipid raft content in smaller EV found in the late SEC fractions, compared to canonical EV eluted in early fractions, consistent with their distinct cellular origin.

Together, these findings further demonstrate that the signal detected by IEL in the late SEC fractions represents atypical EV subpopulations rather than protein contaminant.

### Example 7. IEL Analysis of SEC Fractions of Neuronal Conditioned Medium Detects EV Peak in Early Fractions

To compare size distribution of the EV released in plasma and in tissue culture media, EV were isolated from medium conditioned by iPSC-derived cortical neurons (BrainXell, see Methods). Conditioned medium concentrated by ultrafiltration (see methods), was subjected to SEC (qEV Original, Izon Sciences), the fractions were collected and analyzed by IEL with beads for GAP43, CD171 and CD235 combined with panTSPN detection (FIGS. 7A-7C). Consistent with their neuronal origin, the EV in early fractions showed strong positivity for GAP43 and CD171 (FIGS. 7A, 7B); however, approximately 10% of the total signal was localized the late fractions. In agreement with neuronal differentiation, CD235 IEL beads produced no detectable signal in any of the fractions (FIG. 7C), which additionally confirmed specificity of the method.

In addition, the same qEV fractions were analyzed using CD9, CD63 and CD81 IEL beads followed by GAP43 antibody detection (FIGS. 7D-7F). All tetraspanins eluted in a similar pattern, with a larger peak in early fractions succeeded by the lower dispersed signal in late fractions, suggesting a predominance of larger EV in conditioned media. The CD63 and CD81 signals in the EV from iPSC derived neurons were by an order of magnitude higher than the signal for CD9, suggesting that while NDE size distribution is different between the cell culture and plasma, their TSPN composition remains similar, with relative paucity of CD9-positive EV.

### Example 8. Transmission Electron Microscopy (TEM) of EV of Early and Late SEC Fractions

The results described in Examples 5-7 above exemplify the presence of tissue-specific EV populations in late SEC fractions, hitherto considered to be substantially devoid of EV. For further evaluation of the vesicles identified in early and late SEC fractions, TEM analysis was performed on pooled early (2-5) and late (8-13) fractions generated as described in Example 5 (qEV Original, Izon Sciences).

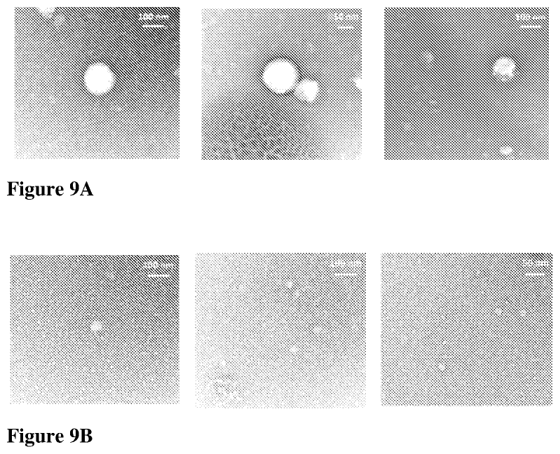

To this end, pooled EV suspensions were fixed in 1% paraformaldehyde overnight. Next, 4 ml of the suspension was loaded onto glow discharged copper mesh Formvar coated carbon grids and allowed to adsorb for approximately 30 seconds. The grids were briefly washed in double-distilled water, followed by staining with 2.5% aqueous uranyl acetate solution, and allowed to dry fully before imaging. Grids were imaged using a FEI Morgagni transmission electron microscope (FEI, Hillsboro, OR) operating at 80 kV and equipped with a Nanosprint5 CMOS camera (AMT, Woburn, MA). The results are depicted in FIGS. 9A-9B (for early and late fractions, respectively, three representative images from each sample).

As can be seen in FIGS. 9A-9B, TEM analyses revealed spherical nano-scale particles in both samples. FIG. 9A depicts EV of 75-125 nm in diameter in fractions 2-5. FIG. 9B exemplifies smaller particles of <30 nm in fractions 8-14, ranging between 15 and 25 nm in diameter.

Together, these findings provide visual demonstration that the signal detected by IEL in the late SEC fractions represents intact EV rather than surface-antigen fragments or other artifacts. Further, these results surprisingly reveal a previously unknown population of neuron-specific EV obtained from plasma samples, having a discrete size distribution profile of remarkably small diameters, herein designated “nano-EV”. Without wishing to be bound by a specific theory or mechanism of action, the properties of these newly identified EV as found herein, including small size, membrane lipid compositions and tissue specificity may provide valuable benefits in therapy and diagnosis.

### Example 9. Multiplexed Measurements of Synaptic Proteins on the Surface of EV from Blood Plasma

The IEL assay was used in this experiment for multiplexed measurement of synaptic proteins on the surface of EV from plasma samples. IEL beads were appended with antibodies against five synaptic markers, as follows: glutamate receptor subunit 2 (GluR2), neurogranin (NRGN), growth-associated protein 43 (GAP43), postsynaptic density (PSD95) and Syntaxin-1 (STXN-1). In addition, IEL beads appended with antibodies against the canonical EV marker CD63 were added to the assay for normalization purposes, when multiple samples with divergent EV content are compared. In this experiment the level of CD63 was used for normalization. However, various alternative normalization methods may be used. For example, the level of a synaptic protein being analyzed may be normalized with respect to the sample volume, the amount of protein and/or the concentration of EVs. The level of a synaptic protein being analyzed may be also normalized with respect to other canonical EV markers, such as CD9, CD81, or a combination of such markers. The level of a synaptic protein being analyzed may also be normalized with respect to general neuronal markers, such as CD171.

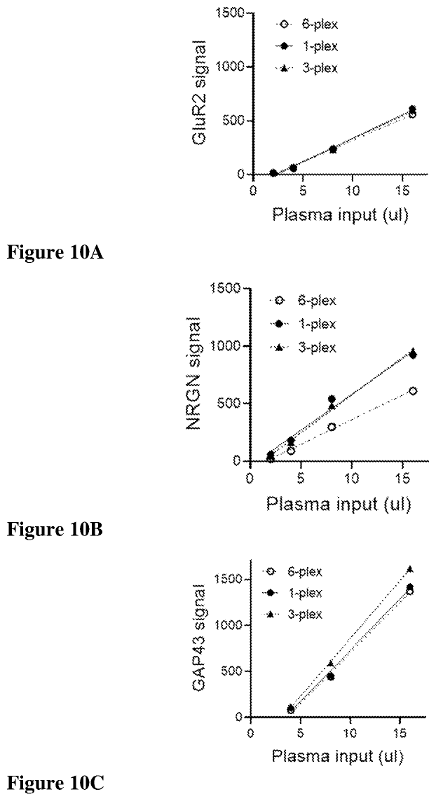

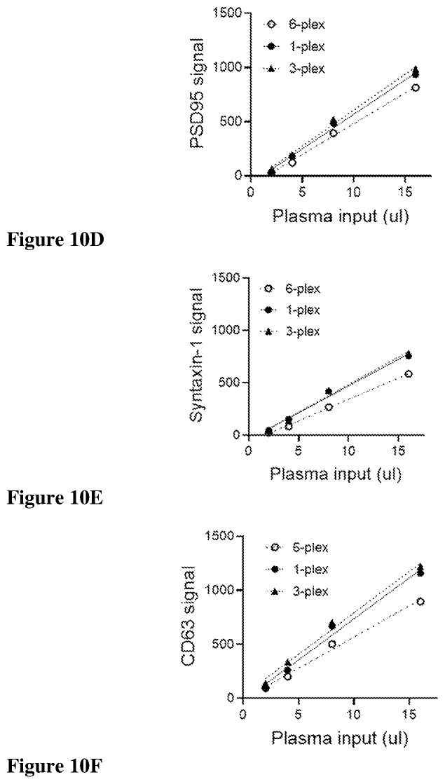

The assay was performed in three formats: single plex (1-plex)—using capture beads for individual analytes; 6-plex—using a combination of capture beads for all six analytes; and 3-plex, wherein two parallel assays with two capture bead combinations (NRGN+GAP43+CD63 and GluR2+PSD95+STXN-1) were carried out. The bead combinations for the 3-plex assays were selected based on the signal strength determined in 1-plex assays. The detection in all experiments was performed using panTSPN antibody cocktail. The results are summarized in FIGS. 10A-10F.

For all analytes that were measured, a clearly detectable concentration-dependent signal was generated, for all assay formats, namely 1-plex, 3-plex and 6-plex. For some analytes the signal strength was reduced in the 6-plex format compared to the 1-plex and 3-plex. However, even when the signal was reduced it was still clear and significant, again demonstrating the multiplexing capability of the assay. Remarkably, the strong positive signals obtained in this assay were achieved using very small amounts of plasma samples, up to 15 μl.

### Example 10. Quantitative Measurements

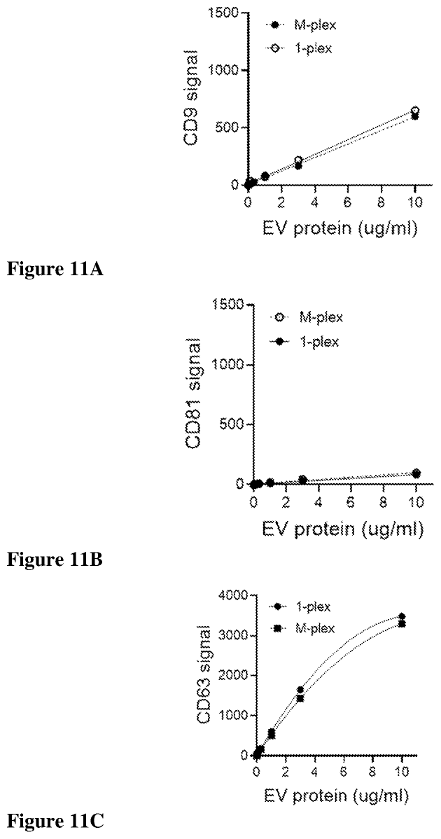

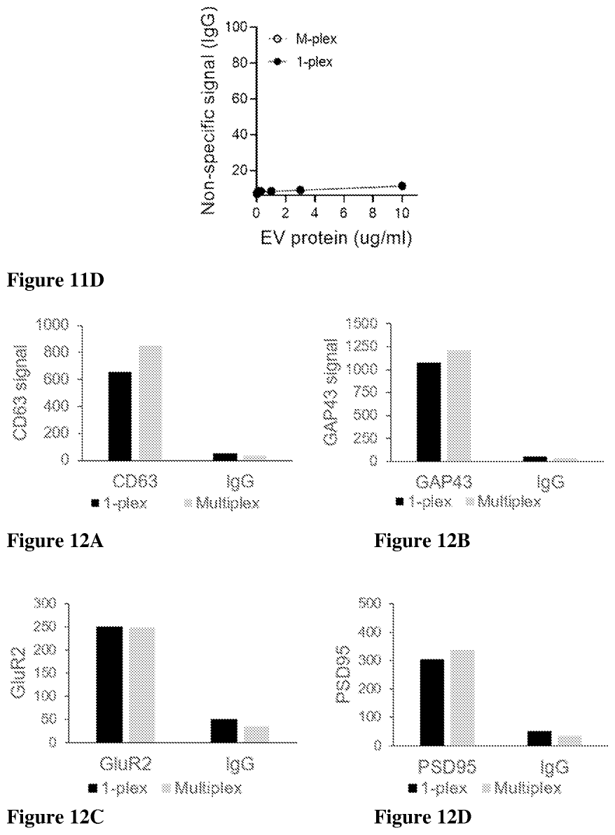

A dilution curve was generated using concentrated purified EV released from cultured glioblastoma cells (U87 cell line, ATCC), as follows: EV were purified from the cultured cells and the protein content (μg/ml) was measured using NanoDrop spectrophotometer (Thermo Fisher Scientific). The purified EV were serially diluted and subjected to IEL assay to measure the EV markers CD9, CD81 and CD63 (FIGS. 11A, 11B and 11C, respectively). The EV makers were measured in single plex (1-plex) and multiplexed (M-plex) formats (see above) with appropriate capture beads and panTSPN antibody cocktail for detection. Additionally, non-specific control beads decorated with isotype control antibodies (IgG) were added to the assay (FIG. 11D). In agreement with published data, U87-derived EV displayed high levels of CD63, lower levels of CD9 and very modest amounts of CD81. All three markers showed clear dilution linearity, thus demonstrating that a linear dependence can be established between the levels of EV surface markers and EV protein, and therefore between the levels of EV surface markers and the number of particles. Such linear correlation can be used subsequently to generate a standard curve, in order to normalize synaptic marker signal per particle number in plasma samples.

### Example 11. Internal Specificity Control

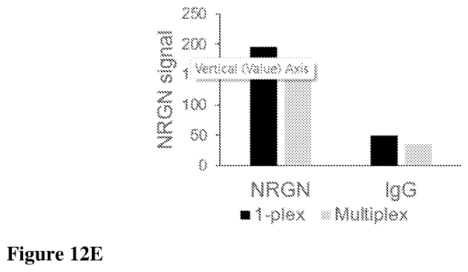

Capture beads decorated with a non-specific isotype control antibody (IgG) were incorporated into the IEL assay, and the assay was performed essentially as described in Example 9. Single plex (1-plex) and multiplexed (3-plex) measurements were compared to ensure the lack of antibody interference. For each analyte, including CD63 (FIG. 12A), GAP43 (FIG. 12B), GluR2 (FIG. 12C), PSD95 (FIG. 12D) and NRGN (FIG. 12E), the specific signal exceeded the non-specific signal (IgG) by at least 10-fold. Such an internal control may be incorporated to all samples, and non-specific background may be subtracted for all analytes, to account for potential matrix effects.

### CONCLUSION

In summary, disclosed herein are highly specific, sensitive and accurate methods and assays, enabling simultaneous identification and evaluation of EV surface markers, wherein the analysis requires only low volume (≤50 μl) of plasma samples, and is applied directly to unprocessed plasma, without the need for EV precipitation or other pre-enrichment steps. The methods and assays as disclosed herein enabled characterization of specific EV populations, revealing unique molecular and physico-chemical properties, allowing differentiation between the various EV populations in plasma. In particular, it was unexpectedly discovered that certain populations of circulating EV, such as those of neuron and macrophage origin, are markedly smaller than the hitherto-postulated average EV size and compared to other EV populations such as erythrocyte-derived EV. These EV populations could also be differentiated by their tetraspanin profiles and by their mRNA and lipid contents. Further, EV populations of neural origin captured from the blood circulation were similar in terms of their marker profiles to EV obtained from cultured neurons, but were remarkably and unexpectedly smaller than those obtained in culture. These newly identified properties may confer an additional advantage in isolating and characterizing specific EV populations of different origins useful in diagnosis and therapy.

In addition, the methods and assays disclosed herein enabled capture and analysis of a plurality of synaptic proteins simultaneously, using very small volumes of plasma samples. The methods and assays disclosed herein provide a more simple and accurate measurement of synaptic proteins in plasma samples, and are highly beneficial for clinical diagnostic purposes.

The foregoing description of the specific embodiments will so fully reveal the general nature of the invention that others can, by applying current knowledge, readily modify and/or adapt for various applications such specific embodiments without undue experimentation and without departing from the generic concept, and therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed chemical structures and functions may take a variety of alternative forms without departing from the invention.

1. An extracellular vesicle (EV) preparation comprising a substantially purified population of EVs isolated from serum or plasma and derived from cells other than blood cells, the population characterized by a size distribution of 10-25 nanometers (nm) in diameter and by a marker profile corresponding to differentiated solid tissue cells.

2. The EV preparation of claim 1, wherein said EVs are 15-25 nm in diameter, 15-20 nm in diameter or 10-15 nm in diameter, and wherein said EVs comprise at least one type of EV selected from neural-derived EV and tissue macrophage-derived EV.

3. (canceled)

4. The EV preparation of claim 2, wherein said EVs comprise neural-derived EVs and tissue macrophage-derived EVs.

5. The EV preparation of claim 1, wherein the population of EVs is characterized by surface display of GAP43, CD171 and/or CD68; and by lack of CD235a.

6. The EV preparation of claim 1, wherein the population of EVs is characterized by a membrane lipid composition according to Table A.

7. The EV preparation of claim 1, wherein the EVs are obtained from the plasma or serum sample by a process comprising a step of size selection for EVs that are under 30 nanometers (nm) in diameter, and isolation of the selected EVs, wherein the step of size selection comprises at least one of size exclusion chromatography, nanoporous membrane filtration, deterministic lateral displacement sorting, dielectrophoretic isolation, acoustic fractionation, and differential centrifugation.

8. (canceled)

9. The EV preparation of claim 7, wherein the process further comprises immunoaffinity purification and/or analysis of the EV population.

10. The EV preparation of claim 1, wherein the EVs are loaded with an exogenous cargo.

11. The EV preparation of claim 10, wherein the exogenous cargo is a therapeutic agent, and/or wherein the EVs are modified to display a targeting agent.

12. (canceled)

13. The EV preparation of claim 1, wherein the purity of the EV population is a pharmaceutical-grade purity.

14. The EV preparation of claim 13, wherein said preparation is formulated in the form of a pharmaceutical composition, further comprising a pharmaceutically acceptable carrier, excipient or diluent.

15. (canceled)

16. A method of delivery of an exogenously loaded therapeutic agent to a target cell or tissue of a subject in need thereof, comprising administering to the subject the EP preparation of claim 11.

17. (canceled)

18. A method of analyzing a plasma or serum sample, comprising:
a. providing a capture system, comprising at least three populations of distinct fluorescence-labeled magnetic microspheres, wherein each microsphere population displays antibodies directed to a distinct target on the surface of an EV population of a distinct cellular origin;
b. providing 1-75 μl of an unprocessed plasma or serum sample, or a corresponding amount of intact EV;
c. incubating the sample with the capture system, under conditions such as to allow specific antigen-antibody binding while substantially maintaining the integrity of the EV membranes, to thereby provide distinct populations of EV-microsphere complexes corresponding to each target;
d. washing the EV-microsphere complexes using a magnetic device, under conditions enabling selective capturing of said complexes;
e. incubating said EV-microsphere complexes with a plurality of detection antibodies, each antibody directed to a distinct marker on the surface of the EV, under conditions such as to allow specific antigen-antibody binding while substantially maintaining the integrity of the EV membranes, wherein the plurality of detection antibodies is labeled by the same fluorescent marker;
f. washing the resulting labeled complexes using a magnetic device to remove excess reagents;
g. subjecting the resulting complexes to a microfluidic device amenable for simultaneously detecting and quantifying fluorescent emission on a plurality of wave lengths, to thereby quantify the fluorescence emission levels and provide a separate assessment of the level of the surface targets corresponding to each of the EV populations; and
h. comparing the quantified levels to control levels;
wherein the method is performed using reagents and under conditions so as to retain said EVs in a substantially intact form, wherein said plurality of detection antibodies is directed to a plurality of diagnostic markers or a plurality of tetraspanin markers.

19-20. (canceled)

21. The method of claim 18, wherein said tetraspanin markers are selected from the group consisting of CD9, CD63 and CD81, and wherein the targets of the capture system comprise at least one neural cell target and targets corresponding to at least two additional cellular origins selected from the group consisting of: bone, lung, tissue macrophage, lung macrophage, muscle, adipocyte, epithelium, endothelium, monocyte, microglia, megakaryocyte, T cell, erythrocyte, liver and oligodendrocyte.

22. The method of claim 18, wherein said plurality of detection antibodies comprises antibodies directed to CD9, antibodies directed to CD63 and antibodies directed to CD81.

23. (canceled)

24. The method of claim 21, wherein the at least one neural cell target is selected from GAP43 and CD171, and wherein the at least two additional targets comprise P2RY12 and CD68.

25. The method of claim 24, wherein the at least two additional targets further comprise CD235a.

26. The method of claim 18, wherein said capture system comprises at least four, at least five, at least six, at least seven or at least eight populations of distinct fluorescence-labeled magnetic microspheres, wherein each microsphere population displays antibodies directed to a distinct target on the surface of an EV population of a distinct cellular origin, and wherein 1-50 μl of unprocessed plasma or serum are provided.

27-36. (canceled)

37. A kit for analyzing EVs from a blood-derived sample, comprising:
i) a capture system, comprising a first population of magnetic microspheres displaying an antibody directed to GAP43 or CD171, and labeled by a first combination of fluorophores, a second population of magnetic microspheres displaying an antibody directed to CD68, labeled by a second combination of fluorophores, and a third population of magnetic microspheres displaying an antibody directed to P2RY12, labeled by a third combination of fluorophores;
ii) a plurality of detection antibodies, each antibody directed to a distinct tetraspanin marker, wherein the plurality of detection antibodies is labeled, directly or indirectly, by the same fluorescent marker; and optionally
iii) reagents for performing said evaluation under conditions so as to retain EVs in a substantially intact form.

38. (canceled)

39. The kit of claim 37, wherein the reagents are selected from the group consisting of:
(i) at least one binding buffer for incubating a sample with the capture system to thereby provide distinct populations of EV-microsphere complexes, the at least one binding buffer characterized by lack of detergents and by the presence of protease and/or phosphatase inhibitors;
(ii) at least one washing buffer, characterized by significantly enhanced salt concentrations compared to the at least one binding buffer; and
(iii) at least one binding buffer and at least one washing buffer as defined in (i) and (ii) above.
